SEQUENCE LISTING (11 sequences)

SEQ ID NO: 1 - protein, 450 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 450 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 3 - protein, 219 aa, synthetic construct
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 451 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 219 aa, synthetic construct; identical to SEQ ID NO: 3

SEQ ID NO: 6 - protein, 5 aa, synthetic construct
SYWMH

SEQ ID NO: 7 - protein, 17 aa, synthetic construct
EIDPSESNTNYNQKFKG

SEQ ID NO: 8 - protein, 12 aa, synthetic construct
GGYDGWDYAIDY

SEQ ID NO: 9 - protein, 16 aa, synthetic construct
RSSQSLAKSYGNTYLS

SEQ ID NO: 10 - protein, 7 aa, synthetic construct
GISNRFS

SEQ ID NO: 11 - protein, 9 aa, synthetic construct
LQGTHQPYT

(19) United States
(12) Patent Application Publication
(10) Pub. No.: US 2026/0234266 A1
(43) Pub. Date: Aug. 13, 2026

(54) HIGH CONCENTRATION A4B7 INTEGRIN ANTIBODY COMPOSITIONS AND METHODS FOR TREATING INFLAMMATORY BOWEL DISEASE

(71) Applicant: Spyre Therapeutics, Inc., Waltham, MA (US)

(72) Inventors: Brian Connolly, Waltham, MA (US); Tapan Das, Waltham, MA (US); Joseph Saweres, Waltham, MA (US)

(73) Assignee: Spyre Therapeutics, Inc., Waltham, MA (US)

(21) Appl. No.: 19/654,019

(22) Filed: Apr. 21, 2026

Related U.S. Application Data
(63) Continuation of application No. PCT/US2025/054823, filed on Nov. 10, 2025.
(60) Provisional application No. 63/719,062, filed on Nov. 11, 2024.

(51) Int. Cl. C07K 16/28; A61K 9/08; A61K 47/10; A61K 47/18

(52) U.S. Cl. CPC C07K 16/2839; A61K 9/08; A61K 47/10; A61K 47/183

(57) ABSTRACT

Provided herein are formulations of α4β7 integrin antibodies and methods of use.

### CROSS-REFERENCE TO RELATED APPLICATIONS

This application is a continuation application of PCT International Application No. PCT/US2025/054823, filed Nov. 10, 2025, which claims the benefit of and priority to U.S. Provisional Application No. 63/719,062, filed Nov. 11, 2024, the entire contents of each of which are incorporated herein by reference.

### SEQUENCE LISTING

This application contains a Sequence Listing which has been submitted electronically in XML format and is hereby incorporated by reference in its entirety. The XML copy, created on Nov. 7, 2025, is titled 220703-011201_PCT_SL.xml and is 12,183 bytes in size.

### TECHNICAL FIELD

Aspects of the present disclosure relate to anti-α4β7 antibody formulations for the treatment of inflammatory bowel disease and methods of treatment with such antibodies.

### BACKGROUND

Integrins are cell-adhesion transmembrane receptors that function as extracellular matrix (ECM)-cytoskeletal linkers and transducers of biochemical and mechanical signals between cells and their environment. Due to their exposure on the cell surface and sensitivity to molecular inhibition, integrins such as α4β7 integrins have been investigated as pharmacological targets for treating various diseases including cancer and inflammatory diseases (e.g., inflammatory bowel disease). However, current integrin therapies have been associated with serious side effects given the role of integrins in important biological processes and/or require multiple and frequent doses to maintain therapeutic efficacy. As such, improved α4β7 therapies are needed.

Inflammatory bowel disease (IBD), which includes conditions such as ulcerative colitis and Crohn's disease, is a chronic inflammatory condition of the gastrointestinal tract. These diseases are characterized by periods of remission and flare-ups, leading to symptoms such as abdominal pain, diarrhea, and weight loss. The exact cause of IBD is not fully understood, but it is believed to involve a combination of genetic, environmental, and immune system factors. Current treatments for IBD aim to reduce inflammation and manage symptoms, but they often come with significant side effects and may not be effective for all patients.

Biologic therapies, including monoclonal antibodies, have emerged as a promising treatment option for IBD. These therapies target specific components of the immune system to reduce inflammation. However, the administration of biologics can be challenging due to factors such as the need for frequent dosing, high injection volumes, and stability issues. There is a need for formulations that allow for lower volume administration while maintaining therapeutic efficacy and stability. Such advancements could improve patient compliance and overall treatment outcomes for individuals suffering from IBD.

### SUMMARY

Aspects of the disclosure provide high concentration formulations of an anti-α4β7 antibody, particularly formulations suitable for use by injection, e.g., with a viscosity of about 35 centiPoise (cP) or lower, which is acceptable in use in autoinjectors; a viscosity of about 25 cP or less, which is useful for a syringe or prefilled syringe for other manual injection. These formulations provide for lower volume administration of anti-α4β7 antibody dose that is effective for treating inflammatory bowel disease, particularly by subcutaneous (SC) administration, and stable anti-α4β7 antibody formulations for both SC and intravenous (IV) administration. In some embodiments, the anti-α4β7 antibody is modified to have an extended half-life in humans in vivo. In some embodiments, the anti-α4β7 antibody comprises (a) a heavy chain polypeptide consisting of an amino acid sequence according to SEQ ID NO: 1, and (b) a light chain polypeptide consisting of an amino acid sequence according to SEQ ID NO: 3. In accordance with embodiments, the formulation includes an anti-α4β7 antibody, particularly a half-life extended modified version of the antibody, at a concentration above about 150 mg/mL. In some embodiments, the formulation comprises a buffer selected from phosphate buffer, Tris, and L-histidine at a concentration of from about 10 mM to about 30 mM; and one or more additives selected from amino acids, amino acid salts, vitamins, chelating agents, and surfactants.

In accordance with embodiments, a formulation of an anti-α4β7 antibody is provided, comprising an anti-α4β7 antibody at a concentration above about 160 mg/mL. In some embodiments, the formulation includes a buffer selected from phosphate buffer, Tris, and L-histidine, with a buffer concentration of from about 10 mM to about 20 mM, arginine or arginine-HCl, a chelating agent, and a surfactant selected from polysorbate-80 (PS80) and poloxamer 188 (P188). The formulation is characterized by a pH ranging from about 6 to about 8 and an osmolality of about 400 to about 600 mOsm/kg, providing for lower volume administration of an antibody dose effective for treating inflammatory bowel disease.

In yet other embodiments, the anti-α4β7 antibody is a half-life extended anti-α4β7 antibody. In some embodiments, the anti-α4β7 antibody concentration is at least about 180 mg/mL. In another embodiment, the anti-α4β7 antibody concentration is at least about 200 mg/mL. In some embodiments, the upper concentration limit is up to about 300 mg/mL of the anti-α4β7 antibody. In some embodiments, the pH of the formulation is about 6.7, and the chelating agent is EDTA, with a concentration of about 0.05 mM. In some embodiments, the surfactant is poloxamer 188 at a concentration of about 0.1% (w/v). In some embodiments, the surfactant is polysorbate 80 at a concentration of about 0.02% (w/v). In some embodiments, the formulation comprises arginine or arginine-HCl. In some embodiments, the arginine concentration is at least about 200 mM. In some embodiments, the arginine concentration is up to about 250 mM. In some embodiments, the osmolality of the formulation is from about 400 mOsm/kg to about 600 mOsm/kg. In another embodiment, the osmolality of the formulation is from about 440 to about 540 mOsm/kg. In some embodiments, the formulation includes L-histidine as the buffer, which maintains pH while avoiding particulates associated with phosphate buffer.

In accordance with other embodiments, a formulation of an anti-α4β7 antibody is provided, comprising an anti-α4β7 antibody at a concentration of at least about 180 mg/mL. The formulation includes a buffer selected from phosphate buffer, Tris, and L-histidine, with a buffer concentration of about 10 mM, arginine or arginine-HCl at a concentration of at least about 125 mM, a chelating agent, and poloxamer 188 at a concentration of about 0.1% (w/v). The formulation is characterized by a pH ranging from about 6 to about 8 and an osmolality of from about 400 mOsm/kg to about 600 mOsm/kg, providing for lower volume administration of an antibody dose effective for treating inflammatory bowel disease.

In accordance with further embodiments, the anti-α4β7 antibody is a half-life extended anti-α4β7 antibody. According to further embodiments, the anti-α4β7 antibody concentration is at least about 200 mg/mL. In some embodiments, the pH of the formulation is about 6.7, and that the chelating agent is EDTA at a concentration of about 0.05 mM. In some embodiments, the arginine concentration is to be up to about 250 mM. In some embodiments, the osmolality of the formulation is from about 440 mOsm/kg to about 540 mOsm/kg. In another embodiment, the osmolality of the formulation is from about 500 to about 600 mOsm/kg.

In some embodiments, the arginine concentration in the formulation is about 250 mM, which maintains acceptable viscosity and improves antibody stability at the antibody concentration of at least about 180 mg/mL.

In some embodiments, formulations have viscosities ranging from about 10 cP to about 80 cP at room temperature (between 20° C. and 25° C.) In some embodiments, formulations have viscosities ranging from about 10 cP to about 50 cP at room temperature. In some embodiments, viscosity is about 30 cP at room temperature. In a specific embodiment, the formulations have a viscosity of from about 10 cP to about 20 cP at 20° C. At about 200 mg/mL or greater antibody concentration, viscosity may range from 30-80 cP.

Viscosity of a solution, along with needle bore size and syringe characteristics, impact the force required to inject a pharmaceutical formulation. The formulations of the disclosure have viscosities that, in conjunction with needle size, ensure acceptable glide force properties for injection. In some embodiments, average glide force ranges from 12-24 Newtons (N). In some embodiments, maximum glide force ranges from 15-30 N. In some embodiments, formulations with a viscosity of 40-45 cP had an average glide force of about 20 N using a 25 gauge (G) needle.

In some embodiments, the formulation comprises an anti-α4β7 antibody at a concentration of at least about 160 mg/mL; a buffer selected from phosphate buffer, Tris, and histidine at a concentration of from about 10 mM to about 20 mM; and one or more additives selected from amino acids, amino acid salts, chelating agents, and surfactants. In some embodiments, the formulation has a pH ranging from about 6 to about 8, an osmolality ranging from about 400 mOsm/kg to about 600 mOsm/kg, and a viscosity ranging from about 10 cP to about 25 cP at 20° C. In some embodiments, the formulation is heat stable at about 25° C. for at least 6 months. In some embodiments, the anti-α4β7 antibody concentration is at least about 180 mg/mL. In some embodiments, the anti-α4β7 antibody concentration is at least about 200 mg/mL. In some embodiments, the pH ranges from about 6.5 to about 7.5, for example 6.7. In some embodiments, the formulation comprises arginine or arginine-HCl. In some embodiments, the arginine or arginine-HCl concentration is from about 200 mM to about 250 mM. In some embodiments, the arginine or arginine-HCl concentration is at least about 200 mM, or at least about 240 mM. In some embodiments, the chelating agent is EDTA. In some embodiments, the EDTA is present at a concentration of about 0.05 mM. In some embodiments, the surfactant is polysorbate-80 or poloxamer 188. In some embodiments, the surfactant is poloxamer 188.at a concentration of (w/v) 0.1%. In some embodiments, the osmolality ranges from about 440 mOsm/kg to about 540 mOsm/kg. In some embodiments, the osmolality ranges from about 500 mOsm/kg to about 600 mOsm/kg. In some embodiments, the osmolality is about 500 mOsm/kg. In some embodiments, the buffer is L-histidine. In some embodiments, the L-histidine is present at a concentration of about 20 mM.

In some embodiments, the formulation comprising an anti-α4β7 antibody at a concentration of at least about 180 mg/mL; a buffer selected from phosphate buffer, Tris, and histidine at a concentration of from about 10 mM to about 20 mM; and one or more additives selected from amino acids, amino acid salts, chelating agents, and surfactants. In some embodiments, the formulation has a pH ranging from about 6.5 to about 7.5, an osmolality ranging from about 400 mOsm/kg to about 600 mOsm/kg, and a viscosity ranging from about 15 cP to about 25 cP at 20° C. In some embodiments, the pH is 6.7. In some embodiments, the formulation comprises arginine. In some embodiments, the arginine concentration is at least about 200 mM. In some embodiments, the chelating agent is EDTA. In some embodiments, the EDTA is present at a concentration of about 0.05 mM. In some embodiments, the surfactant is poloxamer 188. In some embodiments, the poloxamer 188 is present at a concentration of (w/v) 0.1%. In some embodiments, the osmolality ranges is about 465 mOsm/kg. In some embodiments, the buffer is L-histidine. In some embodiments, the L-histidine is present at a concentration of about 20 mM.

In some embodiments, the formulation is stable at the long-term storage condition of 5° C. for at least 9 months. In some embodiments, the formulation is stable at 25° C. for at least six months.

In some embodiments, the anti-α4β7 antibody comprises a modified Fc region and has an extended half-life compared to an anti-α4β7 antibody comprising a wild-type Fc region. In some embodiments, the anti-α4β7 antibody comprises a heavy chain polypeptide consisting of an amino acid sequence according to SEQ ID NO: 1 and a light chain polypeptide consisting of an amino acid sequence according to SEQ ID NO: 3.

Methods for treating inflammatory bowel disease are provided. In some embodiments, the methods comprise administering the formulations provided herein. In some embodiments, the formulation is administered subcutaneously. In some embodiments, the formulation is administered at a lower volume compared to a formulation comprising an antibody with a wild-type Fc region. In some embodiments, the inflammatory bowel disease is ulcerative colitis or Crohn's disease.

### DETAILED DESCRIPTION

To facilitate an understanding of the present disclosure, a number of terms and phrases are defined below.

The term, “antibody,” as used herein, is understood to mean an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs can be defined. The three CDRs, referred to as CDR1, CDR2, and CDR3, contribute to the antibody binding specificity. Naturally occurring antibodies can be used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies).

The terms, “variable domain” and “variable region,” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

The term, “Fc polypeptide,” as used herein, refers to one of the two polypeptides forming the dimeric Fc domain, i.e., a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM. These classes are also designated α, δ, ε, γ, and μ, respectively. Several of these may be further divided into subclasses (isotypes), e.g., IgG₁, IgG₂, IgG₃, IgG₄, IgA₁, and IgA₂.

The terms, “Fc receptor” and “FcR,” as used herein, refer to a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcγRIIA and FcγRIIB have an immunoreceptor tyrosine-based activation motif (ITAM) and an immunoreceptor tyrosine-based inhibition motif (ITIM), respectively. The term, FcR, also includes the neonatal receptor, FcRn, which is responsible for the transfer of maternal IgGs to the fetus.

The terms, “recipient,” “individual,” “subject,” “host” and “patient”, are used interchangeably and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

The term, “effective amount,” as used herein, refers to the amount of a compound (e.g., a compound of the present disclosure) sufficient to effect beneficial or desired results. An effective amount can be administered in one or more administrations, applications or dosages and is not intended to be limited to a particular formulation or administration route.

The term, “treating,” as used herein, includes any effect, e.g., lessening, reducing, modulating, ameliorating or eliminating, that results in the improvement of the condition, disease, disorder, and the like, or ameliorating a symptom thereof.

The term, “pharmaceutical composition,” as used herein, refers to the combination of an active agent with a carrier, inert or active, making the composition especially suitable for diagnostic or therapeutic use in vivo or ex vivo.

The term, “pharmaceutically acceptable carrier,” as used herein, refers to any of the standard pharmaceutical carriers, such as a phosphate buffered saline solution, water, emulsions (e.g., such as an oil/water or water/oil emulsions), and various types of wetting agents. The compositions also can include stabilizers and preservatives.

The terms, “a” and “an,” as used herein, mean “one or more” and include the plural unless the context is inappropriate.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

The term, “about” a number, as used herein, refers to range including the number and ranging from an acceptable variance, e.g., half an order of magnitude for certain biological process or plus/minus 10% below with respect to chemical or physical values, such as mass or concentration. In other embodiments, “about” a range refers to 10% below the lower limit of the range, spanning to 10% above the upper limit of the range.

The term, “percent (%) identity,” as used herein, refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Compositions

Provided herein are compositions comprising polypeptides that have a binding specificity for human α4β7 integrin. Polypeptides described herein may have specificity for α4β7 and not related integrins including α4β1 and αEβ7. In some embodiments, compositions described herein comprise an anti-α4β7 antibody. In some embodiments, the anti-α4β7 antibody comprises two heavy chain polypeptides, and two light chain polypeptides.

In some embodiments, a heavy chain polypeptide comprises an amino acid sequence having at least 80% sequence identity with an amino acid sequence according to SEQ ID NO: 1 or SEQ ID NO: 2. In some embodiments, a heavy chain polypeptide comprises an amino acid sequence having at least 85% sequence identity with an amino acid sequence according to SEQ ID NO: 1 or SEQ ID NO: 2. In some embodiments, a heavy chain polypeptide comprises an amino acid sequence having at least 90% sequence identity with an amino acid sequence according to SEQ ID NO: 1 or SEQ ID NO: 2. In some embodiments, a heavy chain polypeptide comprises an amino acid sequence having at least 95% sequence identity with an amino acid sequence according to SEQ ID NO: 1 or SEQ ID NO: 2. In some embodiments, a heavy chain polypeptide comprises an amino acid sequence having at least 96% sequence identity with an amino acid sequence according to SEQ ID NO: 1 or SEQ ID NO: 2. In some embodiments, a heavy chain polypeptide comprises an amino acid sequence having at least 97% sequence identity with an amino acid sequence according to SEQ ID NO: 1 or SEQ ID NO: 2. In some embodiments, a heavy chain polypeptide comprises an amino acid sequence having at least 98% sequence identity with an amino acid sequence according to SEQ ID NO: 1 or SEQ ID NO: 2. In some embodiments, a heavy chain polypeptide comprises an amino acid sequence having at least 99% sequence identity with an amino acid sequence according to SEQ ID NO: 1 or SEQ ID NO: 2. In some embodiments, a heavy chain polypeptide comprises an amino acid sequence having 100% sequence identity with an amino acid sequence according to SEQ ID NO: 1 or SEQ ID NO: 2. In some embodiment, a heavy chain polypeptide comprises or consists of an amino acid sequence having 100% sequence identity with an amino acid sequence according to SEQ ID NO: 1.

In some embodiments, a light chain polypeptide comprises an amino acid sequence having at least 80% sequence identity with an amino acid sequence according to SEQ ID NO: 3. In some embodiments, a light chain polypeptide comprises an amino acid sequence having at least 85% sequence identity with an amino acid sequence according to SEQ ID NO: 3. In some embodiments, a light chain polypeptide comprises an amino acid sequence having at least 90% sequence identity with an amino acid sequence according to SEQ ID NO: 3. In some embodiments, a light chain polypeptide comprises an amino acid sequence having at least 95% sequence identity with an amino acid sequence according to SEQ ID NO: 3. In some embodiments, a light chain polypeptide comprises an amino acid sequence having at least 96% sequence identity with an amino acid sequence according to SEQ ID NO: 3. In some embodiments, a light chain polypeptide comprises an amino acid sequence having at least 97% sequence identity with an amino acid sequence according to SEQ ID NO: 3. In some embodiments, a light chain polypeptide comprises an amino acid sequence having at least 98% sequence identity with an amino acid sequence according to SEQ ID NO: 3. In some embodiments, a light chain polypeptide comprises an amino acid sequence having at least 99% sequence identity with an amino acid sequence according to SEQ ID NO: 3. In some embodiments, a light chain polypeptide comprises an amino acid sequence having 100% sequence identity with an amino acid sequence according to SEQ ID NO: 3.

In some embodiments, compositions described herein comprise a heavy chain polypeptide comprising an amino acid sequence that is at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 1 or SEQ ID NO: 2, and a light chain polypeptide comprising an amino acid sequence that is at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 3. In some embodiments, a ratio of the heavy chain polypeptide and the light chain polypeptide in the compositions described herein is 1:1.

TABLE 1
Sequences of polypeptide

| SEQ | |
|---|---|
| ID NO | Sequence |
| 1 | QVQLVQSGAEVKKPGASVKVSCKGSGYTFTSYWMHWVRQAPGQRLEWI |
| | GEIDPSESNTNYNQKFKGRVTLTVDISASTAYMELSSLRSEDTAVYYCARG |
| | GYDGWDYAIDYWGQGTLVTVSSASTKGPSVFPLAPSSKSTSGGTAALGCL |
| | VKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELAGAPSVFLFPPKP |
| | KDTLYITREPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPG |
| 2 | QVQLVQSGAEVKKPGASVKVSCKGSGYTFTSYWMHWVRQAPGQRLEWI |
| | GEIDPSESNTNYNQKFKGRVTLTVDISASTAYMELSSLRSEDTAVYYCARG |
| | GYDGWDYAIDYWGQGTLVTVSSASTKGPSVFPLAPSSKSTSGGTAALGCL |
| | VKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELAGAPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPV |
| | LDSDGSFFLYSKLTVDKSRWQQGNVFSCSVLHEALHSHYTQKSLSLSPG |
| 3 | DVVMTQSPLSLPVTPGEPASISCRSSQSLAKSYGNTYLSWYLQKPGQSPQL |
| | LIYGISNRFSGVPDRFSGSGSGTDFTLKISRVEAEDVGVYYCLQGTHQPYTF |
| | GQGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQ |
| | GLSSPVTKSFNRGEC |

In some embodiments, compositions described herein comprise a heavy chain polypeptide comprising an amino acid sequence having at least 80% sequence identity with an amino acid sequence according to SEQ ID NO: 1 or SEQ ID NO: 2; and a light chain polypeptide comprising an amino acid sequence having at least 80% sequence identity with an amino acid sequence according to SEQ ID NO: 3. In some embodiments, a composition comprises a heavy chain polypeptide comprising an amino acid sequence having at least 85% sequence identity with an amino acid sequence according to SEQ ID NO: 1 or SEQ ID NO: 2; and a light chain polypeptide comprising an amino acid sequence having at least 85% sequence identity with an amino acid sequence according to SEQ ID NO: 3. In some embodiments, a composition comprises a heavy chain polypeptide comprising an amino acid sequence having at least 90% sequence identity with an amino acid sequence according to SEQ ID NO: 1 or SEQ ID NO: 2; and a light chain polypeptide comprising an amino acid sequence having at least 90% sequence identity with an amino acid sequence according to SEQ ID NO: 3. In some embodiments, a composition comprises a heavy chain polypeptide comprising an amino acid sequence having at least 95% sequence identity with an amino acid sequence according to SEQ ID NO: 1 or SEQ ID NO: 2; and a light chain polypeptide comprising an amino acid sequence having at least 95% sequence identity with an amino acid sequence according to SEQ ID NO: 3. In some embodiments, a composition comprises a heavy chain polypeptide comprising an amino acid sequence having at least 96% sequence identity with an amino acid sequence according to SEQ ID NO: 1 or SEQ ID NO: 2; and a light chain polypeptide comprising an amino acid sequence having at least 96% sequence identity with an amino acid sequence according to SEQ ID NO: 3. In some embodiments, a composition comprises a heavy chain polypeptide comprising an amino acid sequence having at least 97% sequence identity with an amino acid sequence according to SEQ ID NO: 1 or SEQ ID NO: 2; and a light chain polypeptide comprising an amino acid sequence having at least 97% sequence identity with an amino acid sequence according to SEQ ID NO: 3. In some embodiments, a composition comprises a heavy chain polypeptide comprising an amino acid sequence having at least 98% sequence identity with an amino acid sequence according to SEQ ID NO: 1 or SEQ ID NO: 2; and a light chain polypeptide comprising an amino acid sequence having at least 98% sequence identity with an amino acid sequence according to SEQ ID NO: 3. In some embodiments, a composition comprises a heavy chain polypeptide comprising an amino acid sequence having at least 99% sequence identity with an amino acid sequence according to SEQ ID NO: 1 or SEQ ID NO: 2; and a light chain polypeptide comprising an amino acid sequence having at least 99% sequence identity with an amino acid sequence according SEQ ID NO: 3. In some embodiments, a composition comprises a heavy chain polypeptide comprising an amino acid sequence according to SEQ ID NO: 1 or SEQ ID NO: 2; and a light chain polypeptide comprising an amino acid sequence according to SEQ ID NO: 3.

In some embodiments, compositions described herein comprise a heavy chain polypeptide consisting of an amino acid sequence according to SEQ ID NO: 1; and a light chain polypeptide comprising an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 80% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 85% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 90% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 95% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 96% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 97% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 98% sequence identity with an amino acid sequence according to SEQ ID NO: 3. In some embodiments, a composition comprises a heavy chain polypeptide consisting of an amino acid sequence according to SEQ ID NO: 1; and a light chain polypeptide comprising an amino acid sequence having at least 99% sequence identity with an amino acid sequence according SEQ ID NO: 3.

### Antibodies

In some embodiments, compositions described herein comprise anti-α4β7 antibodies. Accordingly, in some embodiments, anti-α4β7 antibodies described herein comprise a heavy chain amino acid sequence, wherein the heavy chain amino acid sequence comprises HCDR1, HCDR2, and HCDR3. In some embodiments, anti-α4β7 antibodies described herein further comprise a light chain amino acid sequence, wherein the light chain amino acid sequence comprises LCDR1, LCDR2, and LCDR3. Accordingly, in some embodiments, antigen-binding sites of anti-α4β7 antibodies described herein comprise HCDR1, HCDR2, HCDR3, LCDR1, LCDR2, and LCDR3 disclosed in Table 2.

TABLE 2
CDR sequences

| SEQ ID NO | Name | Sequence |
|---|---|---|
| 6 | HCDR1 | SYWMH |
| 7 | HCDR2 | EIDPSESNTNYNQKFKG |
| 8 | HCDR3 | GGYDGWDYAIDY |
| 9 | LCDR1 | RSSQSLAKSYGNTYLS |
| 10 | LCDR2 | GISNRFS |
| 11 | LCDR3 | LQGTHQPYT |

In some embodiments, anti-α4β7 antibodies described herein comprises two heavy chain polypeptides, each independently comprises an amino acid sequence having at least 80%, at least 85%, at least 90%, at least 95%, at least 96%, at least 97%, at least 98%, at least 99% or 100% sequence identity with an amino acid sequence of SEQ ID NO: 1. In some embodiments, anti-α4β7 antibodies described herein comprise two identical heavy chain polypeptides. In some embodiments, anti-α4β7 antibodies described herein comprise two non-identical heavy chain polypeptides.

In some embodiments, anti-α4β7 antibodies described herein comprises two light chain polypeptides, each independently comprises an amino acid sequence having at least 80%, at least 85%, at least 90%, at least 95%, at least 96%, at least 97%, at least 98%, at least 99% or 100% sequence identity with an amino acid sequence of SEQ ID NO: 3. In some embodiments, anti-α4β7 antibodies described herein comprise two identical light chain polypeptides. In some embodiments, anti-α4β7 antibodies described herein comprise two non-identical light chain polypeptides.

In some embodiments, an anti-α4β7 antibody (Ab001) comprises a heavy chain polypeptide consisting of an amino acid sequence according to SEQ ID NO: 1; and a light chain consisting of an amino acid sequence consisting of SEQ ID NO: 3.

In some embodiments, anti-α4β7 antibodies described herein can have properties as described in PCT Application No. PCT/US2024/031569, filed May 30, 2024, which is herein incorporate by reference in its entirety for all purposes.

In some embodiments, anti-α4β7 antibodies described herein can have the properties as provided in Table 3. Viscosity is commonly expressed in units of centiPoise, cP, which is equivalent to milliPascal-second, mPa*S.

TABLE 3
Properties of anti-α4β7 antibody

| Property | Range | Exemplary Value |
|---|---|---|
| Solubility (30° C.) | 100 mg/mL -300 mg/mL | 200 mg/mL |
| Solubility (5° C.) | 90 mg/mL -270 mg/mL | 206.5 mg/mL |
| Viscosity | 6-16 mPa*s (150 mg/mL, | 11.1 mPa*s (161 mg/ |
| | 25° C.) | mL, 25° C.) |
| pI | 7.5-7.9 | 7.7 |
| Expression titer | 1-10 mg/mL | 4.7 mg/mL |

Anti-α4β7 antibodies described herein can have the following N-glycan profiling (determined using enzymatically released N-glycans, labeled and monitored by hydrophilic interaction liquid chromatography (HILIC) coupled with a fluorescence detector (FLD)) as provided in Table 4.

TABLE 4
N-glycan profiling of anti-α4β7 antibody

| Glycan | Range | Exemplary Value |
|---|---|---|
| G0F | 40-75% | 60.4% (sample 1), |
| Glycan | Range | |
| | | 55.1% (sample 2) |
| GIF | 10-35 % | 16.6% (sample 1) |
| | | 18.8% (sample 2) |
| Man5 | 1-15% | 7.3% (sample 1) |
| | | 8.9% (sample 2) |

### Fc Modifications

Provided herein are anti-α4β7 antibodies comprising modified Fc regions, also referred to as modified anti-α4β7 antibodies. Unless otherwise specified herein, numbering of amino acid residues in the Fc region or constant region is according to the EU numbering system, also called the EU index.

In some embodiments, anti-α4β7 antibodies comprise a modified Fc comprising one or more modifications. In some embodiments, the one or more modifications extended half-life of anti-α4β7 antibodies relative to anti-α4β7 antibodies with wildtype Fc regions. In some embodiments, the one or more modifications promote selective binding of Fc-gamma receptors.

In some embodiments, one or more modifications in the modified Fc is selected from the group consisting of: S298A, E333A, K334A, K326A, F243L, R292P, Y300L, V305I, P396L, F243L, R292P, Y300L, L235V, P396L, F243L, S239D, 1332E, A330L, S267E, L328F, D265S, S239E, K326A, A327H, G237F, K326E, G236A, D270L, H268D, S324T, L234F, N325L, V266L, and S267D. In some embodiments, one or more modifications in the modified Fc is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W.

In some embodiments, a modified Fc comprises a specific combination of amino acid substitutions selected from the group consisting of: L234A/L235A; V234A/G237A; L235A/G237A/E318A; S228P/L236E; H268Q/V309L/A330S/A331S; C220S/C226S/C229S/P238S; C226S/C229S/E3233P/L235V/L235A; L234F/L235E/P331S; C226S/P230S; L234A/G237A; and L234A/L235A/G237A; L234A/L235A/P329G.

In some embodiments, a modified Fc comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LA); M428L/N434A; L234A/G237A (LAGA); L234A/L235A/G237A; L234A/L235A/P329G; D265A/YTE; LALA/YTE; LAGA/YTE; LALAGA/YTE; LALAPG/YTE; N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS; SP/DHS; SPLE/DHS; N297A/LA; D265A/LA; LALA/LA; LAGA/LA; LALAGA/LA; LALAPG/LA; N297A/N434A; D265A/N434A; LALA/N434A; LAGA/N434A; LALAGA/N434A; LALAPG/N434A; N297A/N434W; D265A/N434W; LALA/N434W; LAGA/N434W; LALAGA/N434W; LALAPG/N434W; N297A/DQ; D265A/DQ; LALA/DQ; LAGA/DQ; LALAGA/DQ; LALAPG/DQ; N297A/DW; D265A/DW; LALA/DW; LAGA/DW; LALAGA/DW; LALAPG/DW; N297A/YD; D265A/YD; LALA/YD; LAGA/YD; LALAGA/YD; LALAPG/YD; N297A/QVV; D265A/QVV; LALA/QVV; LAGA/QVV, LALAGA/QVV; LALAPG/QVV; DDRVV; N297A/DDRVV; D265A/DDRVV; LALA/DDRVV; LAGA/DDRVV; LALAGA/DDRVV; and LALAPG/DDRVV. In some embodiments, a modified Fc comprises a specific combination of amino acid substitutions selected from the group consisting of M428L/N434S (LS) and M252Y/S254T/T256E (YTE). In some embodiments, a modified Fc comprises M428L/N434S (LS) modifications. In some embodiments, a modified Fc comprises M252Y/S254T/T256E (YTE) modifications. In some embodiments, M252Y/S254T/T256E (YTE) modifications in Fc region extends half-life of anti-α4β7 antibodies relative to anti-α4β7 antibodies having wild-type Fc regions.

In some embodiments, an Fc of anti-α4β7 antibodies described herein lacks a C-terminal lysine residue (e.g., Ab001), the lack of which can decrease pI. In some embodiments, a composition comprising an anti-α4β7 antibody, wherein the Fc lacks C-terminal lysine residue (e.g., Ab001), has a homogeneous charge profile as compared to a composition comprising an anti-α4β7 antibody, wherein the Fc comprises a C-terminal lysine residue. While the lack of C-terminal lysine residue can decrease solubility or decrease recombinant production yield, truncation of C-terminal lysine in the anti-α4β7 antibody described herein yields a highly soluble protein and allows a relatively high production yield of anti-α4β7 antibodies. In particular, Ab001 achieved surprising high yields (greater than 4.5 g/L) and high solubility (greater than 150 g/L).

In some embodiments, an anti-α4β7 antibody that comprises a heavy chain consisting of an amino acid sequence according to SEQ ID NO: 1; and a light chain comprising an amino acid sequence according to SEQ ID NO: 3 (e.g., Ab001) may have increased FcRn binding, while exhibiting satisfactory bioavailability when an anti-α4β7 antibody is administered subcutaneously.

In some embodiments, the anti-α4β7 antibodies described herein include modifications to improve its ability to mediate effector function. Such modifications are known in the art and include afucosylation, or engineering of the affinity of the Fc towards an activating receptor, mainly FCGR3a for antibody-dependent cellular cytotoxicity (ADCC), and towards C1q for complement-dependent cytotoxicity (CDC).

In some embodiments, an antibody provided herein comprises an IgG1 domain with reduced fucose content at position Asn 297 (EU numbering) compared to a naturally occurring IgG1 domain. Such Fc domains are known to have improved ADCC. In some embodiments, such antibodies do not comprise any fucose at position Asn 297 (EU numbering).

In some embodiments, an anti-α4β7 antibody provided herein comprises glycan moieties. In some embodiments, an anti-α4β7 antibody provided herein comprises an IgG1 domain with high total fucose. The major N-glycan types are GOF and GIF. In some embodiments, an anti-α4β7 antibody of the disclosure has one or more of the following N-glycan profiling: (a) a proportion of GOF can be greater than about 40% (e.g., from about 40% to about 75%, from about 40% to about 45%, from about 45% to about 50%, from about 50% to about 55%, from about 55% to about 60%, from about 60% to about 65%, from about 65% to about 70%, from about 70% to about 75%); (b) a proportion of GIF can range from about 10% to about 35% (e.g., from about 10% to about 15%, from about 15% to about 20%, from about 20% to about 25%, from about 25% to about 30%, from about 30% to about 35%); (c) a proportion of Man5 can be less than about 20% (e.g., range from about 1% to about 15%, from about 1% to about 5%, from about 5% to about 10%, from about 10% to about 15%).

In some embodiments, the anti-α4β7 antibodies described herein comprise an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330.

In some embodiments, the anti-α4β7 antibodies described herein comprise an Fc region with at least one galactose residue in the oligosaccharide attached to the Fc region. Such antibody variants may have improved CDC function.

In some embodiments, the anti-α4β7 antibodies described herein comprise one or more alterations that improve or diminish C1q binding and/or CDC.

In some embodiments, an Fc region, as described herein, comprises one or more amino acid substitutions. In some embodiments, the one or more substitutions can increase in one or more of antibody half-life, ADCC activity, ADCP activity, or CDC activity relative to an Fc region without the one or more substitutions. In some embodiments, the one or more amino acid substitutions result in increased antibody half-life at pH 6.0 compared to an antibody comprising a wild-type Fc region. In some embodiments, an antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region.

In some embodiments, an Fc region comprises one or more amino acid substitutions, wherein the one or more substitutions can decrease one or more of ADCC activity, ADCP activity, or CDC activity compared with an Fc region without the one or more substitutions.

In some embodiments, an Fc region binds an Fcγ Receptor selected from the group consisting of: FcγRI, FcγRIIa, FcγRIIb, FcγRIIc, FcγRIIIa, and FcγRIIIb. In some embodiments, an Fc region binds an Fcγ Receptor with higher affinity at pH 6.0 compared to an antibody comprising a wild-type Fc region.

In some embodiments, the anti-α4β7 antibodies described herein comprise an extended half-life (i.e., serum half-life). In some embodiments, the anti-α4β7 antibodies described herein comprise a half-life of at least about 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 94, 96, 100 or more than 100 days in humans. In some embodiments, the anti-α4β7 antibodies described herein comprise an average serum half-life of greater than about 60 days in humans. In some embodiments, the anti-α4β7 antibodies described herein comprise an average serum half-life of greater than about 70 days in humans. In some embodiments, the anti-α4β7 antibodies described herein comprise an average serum half-life of greater than about 75 days in humans. In some embodiments, the anti-α4β7 antibodies described herein comprise an average serum half-life of greater than about 80 days in humans. In some embodiments, the anti-4β7 antibodies described herein comprise an average serum half-life of greater than about 85 days in humans. In some embodiments, the anti-α4β7 antibodies described herein comprise an average serum half-life of greater than about 90 days in humans. In some embodiments, the anti-α4β7 antibodies described herein comprise an average serum half-life of greater than about 100 days in humans.

In some embodiments, the anti-α4β7 antibodies described herein have a half-life that is at least 20% longer than a comparator antibody. In some embodiments, the comparator antibody comprises the same complementarity determining regions and variable regions but different Fc regions. In some embodiments, half-life of the anti-α4β7 antibodies described herein is at least 30% longer, at least 40% longer, at least 50% longer, at least 60% longer, at least 70% longer, at least 80% longer, at least 90% longer, at least 95% longer, at least 100% longer, at least 150% longer, at last 200% longer, at least 250% longer, at least 300% longer or at least 350% longer than half-life of the comparator antibody. In some embodiments, half-life of the anti-α4β7 antibodies described herein is longer than half-life of the comparator antibody by at least 2-fold, at least 3-fold, at least 4-fold, at least 5-fold, at least 6-fold, at least 7-fold, at least 8-fold, at least 9-fold, or at least 10-fold. In some embodiments, half-life of the anti-α4β7 antibodies described herein is longer than half-life of the comparator antibody (e.g., vedolizumab) by at least 4-fold. In some embodiments, the comparator antibody comprises a heavy chain consisting of an amino acid sequence according to SEQ ID NO: 4; and a light chain consisting of an amino acid sequence according to SEQ ID NO: 5.

In some embodiments, the anti-α4β7 antibodies described herein bind α4β7 with a K_{D} of ≤20 nM, ≤15 nM, ≤10 nM, ≤9 nM, ≤8 nM, ≤7 nM, ≤6 nM, ≤5 nM, ≤4 nM, ≤3 nM, ≤2 nM, ≤1 nM, ≤0.9 nM, ≤0.8 nM, ≤0.7 nM, ≤0.6 nM, ≤0.5 nM, ≤0.4 nM, ≤0.3 nM, ≤0.2 nM, ≤0.1 nM, ≤90 μM, ≤80 μM, ≤70 μM, ≤60 μM, ≤50 μM, ≤40 μM, ≤30 μM, ≤20 μM, or ≤10 μM. For example, in some embodiments, a first antigen-binding site binds α4β7 with a K_{D} in a range of about 10 pM-about 1 nM, about 10 pM-about 0.9 nM, about 10 pM-about 0.8 nM, about 10 pM-about 0.7 nM, about 10 pM-about 0.6 nM, about 10 pM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 0.5 nM-about 9 nM, about 0.5 nM-about 8 nM, about 0.5 nM-about 7 nM, about 0.5 nM-about 6 nM, about 0.5 nM-about 5 nM, about 0.5 nM-about 4 nM, about 0.5 nM-about 3 nM, about 0.5 nM-about 2 nM, about 0.5 nM-about 1 nM, about 1 nM-about 10 nM, about 1 nM-about 9 nM, about 1 nM-about 8 nM, about 1 nM-about 7 nM, about 1 nM-about 6 nM, about 1 nM-about 5 nM, about 1 nM-about 4 nM, about 1 nM-about 3 nM, about 1 nM-about 2 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM. In some embodiments, affinity of anti-α4β7 antibody to α4β7 is measured by SPR. In some embodiments, affinity of anti-α4β7 antibody to α4β7 is measured by BLI. In some embodiments, affinity of anti-α4β7 antibody to α4β7 is measured by KinExA.

### Methods of Treatment and Dosage Regimens

Described herein, in some embodiments, are methods of treating a disease or disorder in a human subject in need thereof, the method comprising subcutaneously or intravenously administering to the human subject an effective amount of an anti-α4β7 antibody formulation disclosed herein.

Provided herein are methods of treating a disease or disorder in a human subject in need thereof comprising administering to the human subject one or more initial doses comprising an effective amount of an anti-α4β7 antibody (also referred to herein as “induction dose”, “induction regimen” or “induction therapy”) and one or more subsequent doses comprising an effective amount of the anti-α4β7 antibody (also referred to herein as “maintenance dose”).

In some embodiments, a single or more than one induction dose is administered, followed by maintenance doses. The disclosure contemplates one, two, three, or more induction doses. In some embodiments, divided induction doses are administered one week apart, two weeks apart, three weeks apart, or four weeks apart. For example, in some embodiments, a second induction dose is administered four weeks from when the first induction dose was administered. In some embodiments, maintenance dose is administered every 8, 9, 10, 11, 12, 13, 14, 15, 16, 17, 18, 20, 21, 22, 23, 24, 25, 26 or more weeks. The induction dosing can involve administration of a higher dose than a maintenance dose. Alternatively, the induction dosing amount is the same as the maintenance dosing amount.

In some embodiments, methods described herein comprise administering a total induction dose of at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 800 mg, at least about 900 mg or at least about 1000 mg of an anti-α4β7 antibody in a single/divided dose to a human subject in need thereof. In some embodiments, methods described herein comprise administering a total induction dose of at least about 100 mg of an anti-α4β7 antibody in a single or divided dose to a human subject in need thereof. In some embodiments, methods described herein comprise administering a total induction dose of at least about 300 mg of an anti-α4β7 antibody in a single/divided dose to a human subject in need thereof. In some embodiments, methods described herein comprise administering a total induction dose of at least about 600 mg of an anti-α4β7 antibody in a single/divided dose to a human subject in need thereof. In some embodiments, methods described herein comprise administering a total induction dose of at least about 1000 mg of an anti-α4β7 antibody in a single/divided dose to a human subject in need thereof. In some embodiments, an induction dose can be administered intravenously or subcutaneously. In some embodiments, at least one of divided induction doses can be administered intravenously or subcutaneously.

In some embodiments, methods described herein comprise administering a maintenance dose of anti-α4β7 antibody at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26 weeks or more (e.g., from 4 weeks to 6 weeks, from 6 weeks to 8 weeks, from 8 weeks to 10 weeks, from 10 weeks to 12 weeks, from 12 weeks to 14 weeks, from 14 weeks to 16 weeks, from 16 weeks to 18 weeks, from 18 weeks to 20 weeks, from 20 weeks to 22 weeks, from 22 weeks to 24 weeks, from 24 weeks to 26 weeks or more) after the administration of the final induction dose. In some embodiments, the maintenance dose is lower than the induction dose (e.g., twice, three times or more lower than the induction dose). In some embodiments, the maintenance dose is the same as the induction dose. A maintenance dose can be administered intravenously or subcutaneously.

In some embodiments, methods described herein comprise administering a maintenance dose of at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least 7 about 00 mg, at least about 800 mg, at least about 900 mg or at least about 1000 mg of an anti-α4β7 antibody in a single dose to a human subject in need thereof following administration of the final induction dose. In some embodiments, methods described herein comprise administering a maintenance dose of at least 100 mg of an anti-α4β7 antibody in a single dose to a human subject in need thereof following administration of the final induction dose. In some embodiments, methods described herein comprise administering a maintenance dose of at least 300 mg of an anti-α4β7 antibody in a single dose to a human subject in need thereof following administration of the final induction dose. In some embodiments, methods described herein comprise administering a maintenance dose of at least 600 mg of an anti-α4β7 antibody in a single dose to a human subject in need thereof following administration of the final induction dose. In some embodiments, methods described herein comprise administering a maintenance dose of at least 1000 mg of an anti-α4β7 antibody in a single dose to a human subject in need thereof following administration of the final induction dose.

In some embodiments, a first induction dose, a second induction dose, a first maintenance dose and a second maintenance dose are administered at week 0, week 4, week 14 and week 26, respectively. In some embodiments, each subsequent maintenance dose is administered every three months (Q12W). In some embodiments, each subsequent maintenance dose is administered every six months (Q26W) or Q24W. In some embodiments, each subsequent maintenance dose is administered every twelve months.

In some embodiments, methods described herein comprise administering of at least one dose of an anti-α4β7 antibody. Accordingly, in some embodiments, methods described herein comprise administering of an effective amount of the anti-α4β7 antibody every three months. In some embodiments, methods described herein comprise administering of an effective amount of the anti-α4β7 antibody every six months. In some embodiments, methods described herein comprise administering of at least one dose of the anti-α4β7 antibody every three months. In some embodiments, administering of at least one dose of an anti-α4β7 antibody can be administering of at least one dose of the anti-α4β7 antibody every six months. In some embodiments, administering of at least one dose of an anti-α4β7 antibody can be administering of one dose of the anti-α4β7 antibody every three months. In some embodiments, administering of at least one dose of an anti-α4β7 antibody can be administering of one dose of the anti-α4β7 antibody every six months. In some embodiments, administering of at least one dose of an anti-α4β7 antibody can be administering of one dose of the anti-α4β7 antibody every twelve months.

In some embodiments, the injectable dosage form is an injectable liquid formulation. In some embodiments, the injectable liquid formulation does not comprise citrate.

In some embodiments, the administration of the anti-α4β7 antibody follows a biphasic decline in serum concentration. In some embodiments, repeated dosing of anti-α4β7 antibody does not have a significant influence on clearance (CL) and steady-state volume of distribution (Vss). In some embodiments, pharmacokinetic is not impacted by disease state of a human subject.

Described herein, in certain embodiments, are methods of treating a gastrointestinal inflammatory disease in a human subject in need thereof. As used herein, the term “gastrointestinal inflammatory disease” refers to a disease of the gastrointestinal tract that involves inflammatory pathways. For example, the gastrointestinal inflammatory disease includes, but is not limited to, inflammatory bowel disease, ulcerative colitis (with or without exposure to anti-tumor necrosis factor (anti-TNF), Crohn's disease (including fistulizing Crohn's Disease), chronic pouchitis, collagenous gastritis, microscopic or collagenous colitis, colitis (including immune mediated colitis), sclerosing cholangitis (including in human subjects with underlying inflammatory bowel disease, celiac enteritis, ileitis. In other aspects of the disclosure, provided herein are methods of treating Intestinal Acute Graft Versus Host Disease (aGVHD) (e.g., in human subjects undergoing allogeneic hematopoietic stem cell transplantation (Allo-HSCT)), steroid-refractory acute intestinal graft-versus-host disease (GvHD) (e.g., in human subjects who have undergone Allo-HSCT), Type 1 diabetes (TID) (e.g., with or without anti-TNF pre-treatment), immune checkpoint inhibitor-related colitis in human subjects with genitourinary cancer or melanoma, microscopic colitis (e.g., lymphocytic colitis and collagenous colitis), autoimmune enteropathy, eosinophilic gastroenteritis (e.g. eosinophilic esophagitis), and chronic granulomatous disease.

In some embodiments, the disease is a gastrointestinal inflammatory disease. In some embodiments, the disease is an inflammatory bowel disease. In some embodiments, the inflammatory bowel disease is Crohn's disease. In some embodiments, the inflammatory bowel disease is ulcerative colitis.

In some embodiments, the human subject in need thereof has moderately to severely active ulcerative colitis. The Mayo score is a commonly used disease activity index in UC. The complete Mayo score is composed of four parts: rectal bleeding, stool frequency, physician assessment, and endoscopy appearance, each part being rated from 0 to 3, giving a total score of 0 to 12. In some embodiments, the human subject in need thereof has a Mayo score 6 to 12 with endoscopic sub score ≥2. In some embodiments, administration of the anti-α4β7 antibody results in clinical response defined as a reduction in complete Mayo score of 3 or more points and 30% from baseline, (or a partial Mayo score reduction of 2 or more points and 25% or greater from baseline, if the complete Mayo score was not performed at the visit) with an accompanying decrease in rectal bleeding subscore of 1 or 0. In some embodiments, administration of the anti-α4β7 antibody results in clinical remission, for example results in a complete Mayo score of 2 or less points and no individual subscore greater than 1 point. Alternatively, a modified or adapted Mayo score can be used for inclusion and for the clinical remission endpoint for UC. Clinical remission by the modified or adapted Mayo score is: stool frequency subscore 0 or 1 and not greater than baseline, rectal bleeding subscore of 0, and endoscopic subscore 0 or 1 without friability.

In some embodiments, the human subject in need thereof has moderately to severely active Crohn's disease. The Crohn's disease activity index (CDAI) is a commonly used disease activity index in Crohn's disease (CD) and is calculated from eight independent variables. Crohn's disease “clinical remission” refers to a CDAI score of 150 points or less. The American College of Gastroenterology and the European Crohn's and Colitis Organization define CDAI<150, 250-220, 220-450, and >450 as reflecting remission, mild disease activity, moderate disease activity, and severe disease activity, respectively. In some embodiments, administration of the anti-α4β7 antibody results in clinical response defined as a ≥100-point decrease in the CDAI score from baseline. In some embodiments, administration of the anti-α4β7 antibody results in clinical remission defined as a CDAI score of 150 points or less.

Described herein, in certain embodiments, are methods of treating an inflammatory bowel disease in a human subject in need thereof, the method comprising subcutaneously or intravenously administering to the human subject an effective amount of an anti-α4β7 antibody comprising: a) a heavy chain comprising an amino acid sequence according to SEQ ID NO: 1 or SEQ ID NO: 2; and b) a light chain comprising an amino acid sequence according to SEQ ID NO: 3.

In some embodiments, the inflammatory bowel disease is Crohn's disease or ulcerative colitis. In some embodiments, the inflammatory bowel disease is ulcerative colitis.

Described herein, in certain embodiments, are methods of treating an inflammatory bowel disease in a human subject in need thereof, the method comprising subcutaneously or intravenously administering to the human subject an effective amount of an anti-α4β7 antibody comprising: a) a heavy chain comprising an amino acid sequence according to SEQ ID NO: 1 or SEQ ID NO: 2; and b) a light chain comprising an amino acid sequence according to SEQ ID NO: 3. In some embodiments, the anti-α4β7 antibody is a long acting engineered anti-4β7 antibody, consisting of a heavy chain consisting of an amino acid sequence according to SEQ ID NO: 1, and a light chain consisting of an amino acid sequence according to SEQ ID NO: 3.

In some embodiments, the anti-α4β7 antibody is administered at a dose in a range of about 75 mg to about 1100 mg, about 75 mg to about 750 mg, about 75 mg to about 350 mg, about 75 mg to about 150 mg, about 150 mg to about 1100 mg, about 150 mg to about 750 mg, about 150 mg to about 350 mg, about 350 mg to about 1100 mg, about 350 mg to about 750 mg, or about 750 mg to about 1100 mg. In some embodiments, the anti-α4β7 antibody is administered at a dose of about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1100 mg or about 1200 mg. In some embodiments, the anti-α4β7 antibody is administered at a dose of about 100 mg. In some embodiments, the anti-α4β7 antibody is administered at a dose of about 300 mg. In some embodiments, the anti-α4β7 antibody is administered at a dose of about 600 mg. In some embodiments, the anti-α4β7 antibody is administered at a dose of about 1000 mg.

In some embodiments, administration of the anti-α4β7 antibody is intravenous, intratumoral, intramuscular, subcutaneous, intralesional, intraintestinal, intracolonic, intrarectal, intrapouch, or intraperitoneal. In some embodiments, administration of the anti-α4β7 antibody is through a parenteral route such as intravenous, intramuscular, subcutaneous, intraarterial, or intraperitoneal administration. In some embodiments, administration of the anti-4β7 antibody is intravenous or subcutaneous. In some embodiments, administration of the anti-α4β7 antibody is intravenous. In some embodiments, administration of the anti-α4β7 antibody is subcutaneous.

### Pharmaceutical Compositions

The present disclosure also features pharmaceutical compositions that contain a therapeutically effective amount of the anti-α4β7 antibodies described herein. The pharmaceutical composition can be formulated for use in a variety of drug delivery systems, and in a specific embodiment the formulation is suitable for subcutaneous injection. One or more physiologically acceptable excipients or carriers can also be included in the composition for proper formulation.

In some embodiments, the pharmaceutical composition is a liquid composition. In some embodiments the liquid formulation is an injectable liquid formulation. In some embodiments, the pharmaceutical composition is formulated for subcutaneous administration. In some embodiments, the pharmaceutical composition is formulated for intravenous administration. In some embodiments, the liquid composition is a colorless to brown; in some embodiments, the liquid composition is clear to opalescent. In some embodiments, the liquid composition contains at least 160 mg/mL, or at least 180 mg/mL, or at least 200 mg/mL of an anti-α4β7 antibody. In some embodiments, the liquid composition contains from about 160 mg/mL to about 210 mg/mL of an anti-α4β7 antibody. In some embodiments, the liquid composition contains from about 150 mg/mL to about 160 mg/mL, from about 160 mg/mL to about 170 mg/mL, from about 170 mg/mL to about 180 mg/mL, from about 180 mg/mL to about 190 mg/mL, from about 190 mg/mL to about 200 mg/mL or more of an anti-α4β7 antibody.

In particular embodiments, the formulation provides for administration of a lower volume of the antibody that still provides a dose effective for treating inflammatory bowel diseases such as ulcerative colitis and Crohn's disease. This is achieved by incorporating an anti-α4β7 antibody at a concentration above 160 mg/mL, which can be increased to at least 180 mg/mL or even 200 mg/mL or greater in certain formulations. For example, the formulations can comprise an anti-α4β7 antibody at a concentration of about 160 mg/mL, about 170 mg/mL, about 180 mg/mL, about 190 mg/mL, about 200 mg/mL, about 210 mg/mL or more. The formulation includes a buffer, such as phosphate buffer, Tris, or L-histidine, e.g., at a concentration of from about 10 mM to about 20 mM, which helps maintain the pH within a range of about 6 to about 8, with a specific embodiment at pH 6.7.

Arginine or arginine HCl may be included to reduce viscosity of the formulation, with arginine potentially at a concentration of at least about 200 mM, which can be increased to about 250 mM to maintain acceptable viscosity and improve antibody stability. In some embodiments, the formulation comprises arginine or arginine HCl at a concentration of from about 200 mM to about 210 mM, from about 210 mM to about 220 mM, from about 220 mM to about 230 mM, from about 230 mM to about 240 mM, from about 240 mM to about 250 mM.

A chelating agent, such as EDTA, may be added to stabilize the formulation, with a specific embodiment containing about 0.05 mM EDTA.

A surfactant, such as polysorbate-80 or poloxamer 188, can be included. In some embodiments, poloxamer 188 (P188) prevents degradation and formation of particulates. In a specific embodiment poloxamer 188 is used at a concentration of about 0.1% (w/v).

The osmolality of the formulation ranges from about 400 to about 600 mOsm/kg, ensuring it is within the acceptable range for subcutaneous products. For example, the osmolality of the formulation ranges from about 440 to about 540 mOsm/kg.

In some aspects of the disclosure, the formulation is tailored to meet specific requirements, such as using L-histidine buffer to maintain pH while avoiding particulates associated with phosphate buffer, and/or using a greater arginine concentration to reduce viscosity and/or improve stability at higher antibody concentrations. Arginine can help maintain acceptable viscosity and improve antibody stability. In some embodiments, the formulation includes arginine at a concentration of at least about 200 mM up to about 250 mM to maintain acceptable viscosity and improve antibody stability at even at higher antibody concentrations, such as 180 mg/mL and even 200 mg/mL. The pH of the formulation ranges from about 6 to about 8, with a specific embodiment at pH 6.7 for a formulation with an antibody concentration of 180 mg/mL, which balances lower viscosity and lower chemical degradation. The L-histidine buffer is particularly effective in maintaining pH while avoiding particulates sometimes associated with phosphate buffer. Additionally, the formulation includes a surfactant. In a specific embodiment, the surfactant is poloxamer 188, which reduces degradation and formation of particulates. A chelating agent, such as EDTA, is added to stabilize the formulation. The osmolality of the formulation is controlled to ensure it is within the acceptable range for subcutaneous products, which is about 400-600 mOsm/kg. These components work together to provide a formulation that enables lower volume administration of an antibody dose effective for treating inflammatory bowel disease.

In some embodiments, the pharmaceutical composition is citrate-free. In some embodiments, the pharmaceutical composition is free of phosphate buffer. In some embodiments, the pharmaceutical composition is free of polysorbate, e.g., polysorbate 80.

Pharmaceutical compositions containing an anti-α4β7 antibody disclosed herein can be presented in a dosage unit form and can be prepared by any suitable method. A pharmaceutical composition should be formulated to be compatible with its intended route of administration. Examples of routes of administration are intravenous (IV), subcutaneous (SC), intradermal, inhalation, transdermal, topical, transmucosal, intrathecal and rectal administration. In some embodiments, the anti-α4β7 antibody disclosed herein is administered by intravenously. In some embodiments, the anti-α4β7 antibody disclosed herein is administered intravenously or subcutaneously. In specific embodiments, the anti-α4β7 antibody disclosed herein is administered subcutaneously.

Provided herein are injectable dosage forms comprising the anti-α4β7 antibody. In some embodiments, the injectable dosage form comprises at least about 160 mg/mL, at least about 180 mg/mL, or at least about 200 mg/mL of the anti-α4β7 antibody. For example, the injectable dosage form comprises from at least about 160 mg/mL to at least about 170 mg/mL, from at least about 170 mg/mL to at least about 180 mg/mL, from at least about 180 mg/mL to at least about 190 mg/mL, from at least about 190 mg/mL to at least about 200 mg/mL, from at least about 200 mg/mL to at least about 210 mg/mL or more of the anti-α4β7 antibody.

In some embodiments, the injectable dosage form comprises from about 100 mg to about 1200 mg of the anti-α4β7 antibody. In some embodiments, the injectable dosage form comprises from about 100 mg to about 300 mg of the anti-α4β7 antibody. In some embodiments, the injectable dosage form comprises from about 300 mg to about 600 mg of the anti-α4β7 antibody. In some embodiments, the injectable dosage form comprises from about 600 mg to about 1000 mg of the anti-α4β7 antibody. In some embodiments, the injectable dosage form comprises from about 1000 mg to about 1200 mg of the anti-α4β7 antibody. In some embodiments, the injectable dosage form comprises about 100 mg of the anti-α4β7 antibody. In some embodiments, the injectable dosage form comprises about 300 mg of the anti-α4β7 antibody. In some embodiments, the injectable dosage form comprises about 600 mg of the anti-α4β7 antibody. In other embodiments, the injectable dosage form comprises about 1000 mg of the anti-α4β7 antibody. In other embodiments, the injectable dosage form comprises about 1200 mg of the anti-α4β7 antibody.

An intravenous or subcutaneous drug delivery formulation may be contained in a vial, syringe, pen, or bag. In some embodiments, the bag is connected to a channel comprising a tube and/or a needle. In some embodiments, the formulation is a lyophilized formulation or a liquid formulation. In some embodiments, the formulation is a liquid formulation. Various devices can be used to deliver liquid formulations by subcutaneous route of administration, including on-body infusion devices, autoinjector devices, prefilled syringes, and syringes. Generally, administration time depends on volume and device, and can range from seconds to minutes. The disclosure provides more optimal formulations for faster administration of a lower volume injectable formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration.

A polyol, which acts as a tonicifier and may stabilize the anti-α4β7 antibody, may also be included in the formulation. The polyol is added to the formulation in an amount which may vary with respect to the desired isotonicity of the formulation. In some embodiments, the aqueous formulation is isotonic. The amount of polyol added may also be altered with respect to the molecular weight of the polyol. For example, a lower amount of a polyol (e.g., mannitol) is added, compared to a disaccharide (such as trehalose). In some embodiments, the polyol which is used in the formulation as a tonicity agent is mannitol.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In some embodiments, the formulation may include a surfactant which is a polysorbate. In some embodiments, the formulation may contain poloxamer 188 (P188).

In some embodiments, the pH of the liquid formulation is titrated by addition of a pharmaceutically acceptable acid and/or base. In some embodiments, the pharmaceutically acceptable acid is hydrochloric acid. In some embodiments, the base is sodium hydroxide. In some embodiments, the pH of the liquid formulation is from about 3.5 to about 9, from about 5.5 to about 7.0, for example 6.7.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The specific dose can be a uniform dose for each human subject of about 100 mg, about 150 mg, of about 200 mg, of about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 1000 mg, about 1100 mg, about 1200 mg or more of anti-α4β7 antibody. Alternatively, a human subject's dose can be tailored to the approximate body weight or surface area of the human subject. Other factors in determining the appropriate dosage can include the disease or condition to be treated or prevented, the severity of the disease, the route of administration, and the age, sex and medical condition of the human subject. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual human subject's dosage can be adjusted as the progress of the disease is monitored. Blood levels of the targetable construct or complex in a human subject can be measured to see if the dosage needs to be adjusted to reach or maintain an effective concentration. Pharmacogenomics may be used to determine which targetable constructs and/or complexes, and dosages thereof, are most likely to be effective for a given individual.

### Kits

Provided herein is a kit for treating a disease in a human subject in need thereof comprising: (a) an injectable liquid formulation comprising an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer from about 100 mg to about 1200 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable formulation comprises from at least about 150 mg/mL to at least about 200 mg/mL of the anti-α4β7 antibody. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an injectable liquid formulation comprising an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer at least 100 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable formulation comprises from at least about 150 mg/mL to at least about 200 mg/mL of the anti-α4β7 antibody. In some embodiments, the injectable formulation comprises at least about 160 mg/mL of the anti-α4β7 antibody. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an injectable liquid formulation comprising an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer at least 300 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable formulation comprises from at least about 150 mg/mL to at least about 200 mg/mL of the anti-α4β7 antibody. In some embodiments, the injectable formulation comprises at least about 160 mg/mL of the anti-α4β7 antibody. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an injectable liquid formulation comprising an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer at least 600 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable formulation comprises from at least about 150 mg/mL to at least about 200 mg/mL of the anti-α4β7 antibody. In some embodiments, the injectable formulation comprises at least about 160 mg/mL of the anti-α4β7 antibody. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an injectable liquid formulation comprising an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer at least 1000 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable formulation comprises from at least about 150 mg/mL to at least about 200 mg/mL of the anti-α4β7 antibody. In some embodiments, the injectable formulation comprises at least about 160 mg/mL of the anti-α4β7 antibody. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an injectable liquid formulation comprising from at least about 150 mg/mL to at least about 200 mg/mL of an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) instructions to administer from about 100 mg to about 1200 mg of the anti-α4β7 antibody. In some embodiments, the anti-α4β7 antibody comprises a heavy chain consisting of an amino acid sequence according to SEQ ID NO: 1, and a light chain consisting of an amino acid sequence according to SEQ ID NO: 3. In some embodiments, the injectable liquid formulation is a subcutaneous formulation. In some embodiments, the injectable liquid formulation is an intravenous formulation.

Provided herein is a kit for treating a disease in a human subject in need thereof comprising: (a) an first injectable liquid formulation comprising about 150 mg/mL of an anti-α4β7 antibody, wherein the anti-α4β7 antibody comprises a heavy chain consisting of an amino acid sequence according to SEQ ID NO: 1, and a light chain consisting of an amino acid sequence according to SEQ ID NO: 3, and (b) an second injectable liquid formulation comprising about 180 mg/mL of the anti-α4β7 antibody, and (c) instructions to administer from about 100 mg to about 1200 mg of the anti-α4β7 antibody. In some embodiments, the first injectable liquid formulation is an intravenous formulation. In some embodiments, the second injectable liquid formulation is a subcutaneous formulation.

### Methods of Preparation

The anti-α4β7 antibodies described above can be made using recombinant DNA technology well known to a skilled person in the art and as described in PCT Application No. PCT/US2024/031569, filed May 30, 2024, incorporated herein by reference. For example, one or more isolated polynucleotides encoding the anti-α4β7 antibody can be ligated to other appropriate nucleotide sequences, including, for example, constant region coding sequences, and expression control sequences, to produce conventional gene expression constructs (i.e., expression vectors) encoding the desired anti-α4β7 antibodies. Production of defined gene constructs is within routine skill in the art.

### EXEMPLARY EMBODIMENTS

Embodiment 1: A formulation comprising: an anti-α4β7 antibody at a concentration of at least about 160 mg/mL; a buffer selected from phosphate buffer, Tris, and L-histidine at a concentration of from about 10 mM to about 20 mM; and one or more additives selected from amino acids, amino acid salts, chelating agents, and surfactants; wherein the formulation has a pH ranging from about 6 to about 8, an osmolality ranging from about 400 mOsm/kg to about 600 mOsm/kg, and a viscosity ranging from about 10 cP to about 25 cP at 20° C., and wherein the formulation is heat stable at about 25° C. for at least 6 months.
Embodiment 2: A formulation comprising: an anti-α4β7 antibody at a concentration of at least about 150 mg/mL; a buffer selected from phosphate buffer, Tris, and L-histidine at a concentration of from about 10 mM to about 20 mM; and one or more additives selected from amino acids, amino acid salts, chelating agents, and surfactants; wherein the formulation has a pH ranging from about 6 to about 8, an osmolality ranging from about 400 mOsm/kg to about 600 mOsm/kg, and a viscosity ranging from about 10 cP to about 25 cP at 20° C., and wherein the formulation is heat stable at about 25° C. for at least 6 months.
Embodiment 3: A formulation comprising: an anti-α4β7 antibody at a concentration of at least about 150 mg/mL; about 20 mM L-histidine buffer, about 125 mM Arginine, about 0.05 mM EDTA, about 0.02% (w/v) polysorbate 80, wherein the formulation has a pH about 6, a viscosity of about 13 cP at 20° C., and an osmolality of about 354 mOsm/kg and wherein the formulation is heat stable at about 25° C. for at least 6 months.
Embodiment 4: A formulation comprising: an anti-α4β7 antibody at a concentration of at least about 180 mg/mL; about 20 mM L-histidine buffer, about 200 mM Arginine, about 0.05 mM EDTA, about 0.1% (w/v) Poloxamer 188, wherein the formulation has a pH about 6.7, a viscosity of about 19 cP at 20° C., and an osmolality of about 465 mOsm/kg and wherein the formulation is heat stable at about 25° C. for at least 6 months.
Embodiment 5: The formulation of any one of embodiments 1-4, wherein the formulation is an injectable formulation.
Embodiment 6: A prefilled syringe comprising the injectable formulation of embodiment 5.
Embodiment 7: The injectable formulation of embodiment 5 or the prefilled syringe of embodiment 6 for subcutaneous administration.
Embodiment 8: The formulation of any one of embodiments 1-5 or the prefilled syringe of embodiment 6 for use in the treatment of a gastrointestinal inflammatory disease.
Embodiment 9: The formulation of any one of embodiments 1-5 or the prefilled syringe of embodiment 6 for use in the treatment of inflammatory bowel disease.
Embodiment 10: The formulation of any one of embodiments 1-5 or the prefilled syringe of embodiment 6 for use in the treatment of ulcerative colitis.
Embodiment 11: The formulation of any one of embodiments 1-5 or the prefilled syringe of embodiment 6 for use in the treatment of Crohn's disease.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1. Formulations of High Concentration of Ab001

### Materials and Methods

Screening for the excipient and surfactant strength that help stabilize Ab001 at concentrations above 160 mg/mL was undertaken. In particular, protein stability of formulation candidates was evaluated under stress conditions. Starting material was Ab001 at a concentration of 200 mg/mL protein in 20 mM His buffer, 100 mM Arg HCl, pH 6.5. The study period was 8 Weeks. Up to 8 formulations with good viscosity performance among them were selected and prepared for stability evaluation (Tables 4 and 5).

TABLE 4
Test Formulations

| No. | Conc. | Buffer | pH | Excipient and Surfactant (w/v) |
|---|---|---|---|---|
| F01 | 200 | 10 mM PB | 7.0 | 250 mM Arg-HC1, 0.05 mM EDTA, 0.02% PS80 |
| F02 | | 10 mM PB | 7.5 | 250 mM Arg-HC1, 0.05 mM EDTA, 0.02% PS80 |
| F03 | | | | 150 mM His, 175 mM Niacinamide, 0.05 mM EDTA, 0.02% |
| | | | | PS80 |
| F04 | | | 8.0 | 150 mM His, 175 mM Niacinamide, 0.05 mM EDTA, 0.02% |
| | | | | PS80 |
| F05 | | | | 50 mM His, 250 mM Niacinamide, 0.05 mM EDTA, 0.02% |
| | | | | PS80 |
| F06 | | 10 mM Tris | | 150 mM His, 175 mM Niacinamide, 0.05 mM EDTA, 0.02% |
| | | | | PS80 |
| F07 | 180 | 10 mM His | 6.5 | 200 mM Arg· HC1, 0.05 mM EDTA, 0.02% PS80 |
| F08 | | 10 mM PB | 7.0 | 200 mM Arg· HCI, 0.05 mM EDTA, 0.02% PS80 |

Abbreviations: PB = phosphate buffer; EDTA form used is Edetate disodium dihydrate

TABLE 5
Stability parameters

| Attributes | Condition | Sampling Points and Assay | | |
|---|---|---|---|---|
| Thermal (in vial) | 40° C* | T0 | 2 Weeks | 4 Weeks |
| | | X, Y, S, P, | X, Y, S | X, Y, S, P |
| Thermal (in PFS) | | V, W | X, Y, S, W | X, Y, S, P, W |
| Freeze/Thaw | −70° C. to | | 3 Cycles | 5 Cycles |
| (in vial) | RT | | X | X, Y, S |
| Agitation (in | 100 rpm, | | 1D | 3D |
| PFS) | 25° C. | | X | X, Y, S, W |

| | | |
|---|---|---|
| Control | 2~8° C. | Backup samples |
| | 25° C. | |

X = Appearance, protein concentration, SE-UPLC
Y = Caliper-SDS (NR & R), iCIEF
S = Sub-visible particles (MFI)
P = Potency, PTM (Only for the selected formulation)
V= TmOnset, Osmolality, pH, Viscosity
W = BLGF test only for DP in PFS
Abbreviations: PFS = pre-filled syringe

Results. Data were obtained for stability, osmolality, and viscosity for various formulations is shown at Table 6.

TABLE 6
Stability, Osmolality, and Viscosity Measures

| | | | | | | | | | | | | | | Osmo- | Vis- |
|---|---|---|---|---|---|---|---|---|---|---|---|---|---|---|---|
| | | Excipient | | | | | | Protein Conc. | | | | | | lality | cosity |
| | | and | Appearance | | | | | | 40° C.-2 W | | 40° C.-4 W | | | (mOsm/ | mPa*s- |
| | | Surfactant | | 40° C.-2 W | | 40° C.-4 W | | | In | In | In | In | pH | kg) | 20° C. |
| No. | Buffer | (w/v) | T0 | In Vial | In PFS | In Vial | In PFS | T0 | Vial | PFS | Vial | PFS | T0 | T0 | T0 |
| F01 | 10 mM | 250 mM Arg- | SY, | SY, SO, | SY, SO, | SY, SO, | SY, SO, | 196.0 | 202.2 | 200.2 | 196.1 | 199.8 | 7.1 | 599 | 24.7 |
| | PB, pH | HCl, 0.05 mM | SO, | NEFP | FP | NEFP | FP | | | | | | | | |
| | 7.0 | EDTA, 0.02% | FP | | | | | | | | | | | | |
| | | PS80 | | | | | | | | | | | | | |
| F02 | 10 mM | 250 mM Arg- | SY, | SY, SO | SY, SO, | SY, SO, | SY, SO, | 193.0 | 199.4 | 203.2 | 195.3 | 195.2 | 7.6 | 608 | 20.6 |
| | PB, pH | HCl, 0.05 mM | SO, | NEFP | FP | NEFP | FP | | | | | | | | |
| | 7.5 | EDTA, 0.02% | FP | | | | | | | | | | | | |
| | | PS80 | | | | | | | | | | | | | |
| F03 | | 150 mM His, | SY, | Y, SO, | SY, SO, | Y, SO, | SY, SO, | 210.1 | 216.7 | 218.1 | 213.1 | 215.3 | 7.6 | 361 | 25.1 |
| | | 175 mM | SO, | FP | FP | FP | FP | | | | | | | | |
| | | Niacinamide, | FP | | | | | | | | | | | | |
| | | 0.05 mM EDTA, | | | | | | | | | | | | | |
| | | 0.02% PS80 | | | | | | | | | | | | | |
| F04 | 10 mM | 150 mM His, | SY, | Y, SO, | SY, SO, | Y, SO, | SY, SO, | 206.5 | 218.4 | 217.3 | 211.2 | 215.0 | 8.0 | 344 | 21.0 |
| | PB, pH | 175 mM | SO, | FP | FP | FP | FP | | | | | | | | |
| | 8.0 | Niacinamide, | FP | | | | | | | | | | | | |
| | | 0.05 mM EDTA, | | | | | | | | | | | | | |
| | | 0.02% PS80 | | | | | | | | | | | | | |
| F05 | | 50 mM His, 250 | SY, | SY, SO, | SY, SO, | SY, SO, | SY, SO, | 216.4 | 218.4 | 220.8 | 213.5 | 218.0 | 7.9 | 318 | 22.8 |
| | | mM | SO, | FP | FP | FP | FP | | | | | | | | |
| | | Niacinamide, | FP | | | | | | | | | | | | |
| | | 0.05 mM EDTA, | | | | | | | | | | | | | |
| | | 0.02% PS80 | | | | | | | | | | | | | |
| F06 | 10 mM | 150 mM His, | SY, | Y, SO, | SY, SO, | Y, SO, | SY, SO, | 210.5 | 220.5 | 218.7 | 215.7 | 211.4 | 8.0 | 355 | 22.9 |
| | Tris, pH | 175 mM | SO, | FP | FP | FP | FP | | | | | | | | |
| | 8.0 | Niacinamide, | FP | | | | | | | | | | | | |
| | | 0.05 mM EDTA, | | | | | | | | | | | | | |
| | | 0.02% PS80 | | | | | | | | | | | | | |
| F07 | 10 mM | 200 mM | SY, | SY, SO, | SY, SO, | SY, SO, | SY, SO, | 198.1 | 205.1 | 203.5 | 200.8 | 198.1 | 6.5 | 441 | 34.2 |
| | His, pH | Arg · HCl, 0.05 | SO, | FP | FP | FP | FP | | | | | | | | |
| | 6.5 | mM EDTA, | FP | | | | | | | | | | | | |
| | | 0.02% PS80 | | | | | | | | | | | | | |
| F08 | 10 mM | 200 mM | SY, | SY, SO, | SY, SO, | SY, SO, | SY, SO, | 184.4 | 184.2 | 187.9 | 184.5 | 186.2 | 6.8 | 431 | 19.4 |
| | PB, pH | Arg · HCl, 0.05 | SO, | NEFP | FP | NEFP | EFP | | | | | | | | |
| | 7.0 | mM EDTA, | FP | | | | | | | | | | | | |
| | | 0.02% PS80 | | | | | | | | | | | | | |

C: Colorless;
CL: Clear liquid;
SO: Slightly opalescent;
SY: Slightly yellow;
Y: Yellow;
FP: Free of visible particles;
EFP: Essentially free of visible particles (no more than 3 visible particles were observed);
NEFP: Not essentially free of visible particles (more than 3 visible particles were observed).
Abbreviations: PB = phosphate buffer; His = L-histidine (throughout these Examples); PFS = pre-filled syringe

### Example 2. Viscosity Screening of Formulations of High Concentration of Ab001, e.g., 200 mg/mL

### Materials and Methods

Screening for acceptable viscosity at high Ab001 protein concentration was undertaken. Starting material was Ab001 at a concentration of 200 mg/mL protein in 20 mM His buffer, 100 mM Arg HCl, pH 6.5.

TABLE 7
Test formulations

| | Protein | |
|---|---|---|
| No. | concentration | Formulation buffer |
| F09 | 200 | 10 mM PB, 200 mM Arg. HC1, 0.05 mM EDTA, 0.02% PS80, pH 6.5 |
| F10 | 200 | 10 mM PB, 200 mM Arg· HC1, 2% sucrose, 0.05 mM EDTA, 0.02% |
| | | PS80, pH 6.5 |
| F11 | 200 | 10 mM PB, 200 mM Arg·HC1, 0.05 mM EDTA, 0.02% PS80, 0.1% |
| | | P188, pH 6.5 |
| F12 | 200 | 10 mM PB, 200 mM Arg· HC1, 2% sucrose, 0.05 mM EDTA, 0.02% |
| | | PS80, pH 7.0 |
| F13 | 200 | 10 mM PB, 200 mM Arg· HC1, 2% sucrose, 0.05 mM EDTA, 0.02% |
| | | PS80, 0.1% P188, pH 7.0 |
| F14 | 200 | 10 mM PB, 230 mM Arg· HC1, 0.05 mM EDTA, 0.02% PS80, pH 6.5 |
| F15 | 200 | 10 mM PB, 230 mM Arg· HC1, 2% sucrose, 0.05 mM EDTA, 0.02% |
| | | PS80, pH 6.5 |
| F16 | 200 | 10 mM PB, 230 mM Arg. HC1, 0.05 mM EDTA, 0.02% PS80, 0.1% |
| | | P188, pH 6.5 |
| F17 | 200 | 10 mM His, 200 mM Arg. HC1, 0.05 mM EDTA, 0.02% PS80, pH 6.7 |
| F18 | 200 | 10 mM His, 250 mM Arg. HC1, 0.05 mM EDTA, 0.02% PS80, pH 6.7 |
| F19 | 210 | 10 mM His, 200 mM Arg.HC1, 0.05 mM EDTA, 0.02% PS80, pH 6.7 |
| F20 | 210 | 10 mM His, 250 mM Arg. HC1, 0.05 mM EDTA, 0.02% PS80, pH 6.7 |
| F21 | 200 | 20 mM Histidine, 230 mM Arg-HC1, 0.1% P188, pH 6.8 |
| F22 | 200 | 20 mM Histidine, 230 mM Arg-HC1, 0.1% P188, pH 7.0 |

Abbreviations: PB = phosphate buffer, His = L-histidine

TABLE 8
Measured Attributes

| Attributes | Condition | Sampling Points and Assay | | |
|---|---|---|---|---|
| Thermal | 40° C. | T0 | 1W | 2W |
| | | W, X, Y, Z | W, X | W, X, P |

W = sub-visible particle (MFI)
X = Appearance, protein concentration, SE-UPLC, iCIEF, Caliper (NR & R)
Y= viscosity, osmolality, pH
Z= BLGF (3mL syringe with 25G needle, injection time: 15 s)
P= potency (only for the selected formulation)

Results. Data were obtained for stability, osmolality, and viscosity for various formulations. See Table 9.

TABLE 9
Stability, Osmolality, and Viscosity Measures

| | | | | | | | | | | | Osmo- | Vis- |
|---|---|---|---|---|---|---|---|---|---|---|---|---|
| | | | | | | | | | | | lality | cosity |
| | | | Appearance | | | Protein Conc. | | | | | (mOsm/ | mPa*s- |
| | Protein | | | 40° C.- | 40° C.- | | 40° C.- | 40° C.- | 40- | pH | kg) | 20° C. |
| No. | Conc. | Formulation | T0 | 1 W | 2 W | T0 | 1 W | 2 W | 3 W | T0 | T0 | T0 |
| F09 | 200 | 10 mM PB, 200 mM | SY, SO, | SY, SO, | SY, SO, NEFP | 206.8 | 204.5 | 198.6 | NA | 6.5 | 482 | 49.0 |
| | | Arg · HCl, 0.05 mM EDTA, | FP | NEFP | | | | | | | | |
| | | 0.02% PS80, pH 6.5 | | | | | | | | | | |
| F10 | 200 | 10 mM PB, 200 mM | SY, SO, | SY, SO, | SY, SO, NEFP | 196.7 | 193.6 | 198.1 | NA | 6.6 | 543 | 39.0 |
| | | Arg · HCl, 2% sucrose, 0.05 | FP | NEFP | | | | | | | | |
| | | mM EDTA, 0.02% PS80, | | | | | | | | | | |
| | | pH 6.5 | | | | | | | | | | |
| F11 | 200 | 10 mM PB, 200 mM | SY, SO, | SY, SO, | SY, SO, NEFP | 206.5 | 203.0 | 199.2 | NA | 6.6 | 500 | 47.7 |
| | | Arg · HCl, 0.05 mM EDTA, | FP | FP | | | | | | | | |
| | | 0.02% PS80, 0.1% | | | | | | | | | | |
| | | P188, pH 6.5 | | | | | | | | | | |
| F12 | 200 | 10 mM PB, 200 mM | SY, SO, | SY, SO, | SY, SO, NEFP | 196.4 | 196.6 | 199.8 | NA | 7.0 | 542 | 33.0 |
| | | Arg · HCl, 2% sucrose, 0.05 | FP | NEFP | | | | | | | | |
| | | mM EDTA, 0.02% PS80, | | | | | | | | | | |
| | | pH 7.0 | | | | | | | | | | |
| F13 | 200 | 10 mM PB, 200 mM | SY, SO, | SY, SO, | SY, SO, FP | 196.8 | 194.6 | 198.9 | NA | 7.0 | 543 | 33.1 |
| | | Arg · HCl, 2% sucrose, 0.05 | FP | FP | | | | | | | | |
| | | mM EDTA, 0.02% | | | | | | | | | | |
| | | PS80, 0.1% P188, pH 7.0 | | | | | | | | | | |
| F14 | 200 | 10 mM PB, 230 mM | SY, SO, | SY, SO, | SY, SO, NEFP | 206.4 | 209.8 | 209.7 | NA | 6.7 | 555 | 39.9 |
| | | Arg · HCl, 0.05 mM EDTA, | FP | NEFP | | | | | | | | |
| | | 0.02% PS80, pH 6.5 | | | | | | | | | | |
| F15 | 200 | 10 mM PB, 230 mM | SY, SO, | SY, SO, | SY, SO, NEFP | 200.9 | 198.1 | 198.8 | NA | 6.6 | 636 | 36.6 |
| | | Arg · HCl, 2% sucrose, 0.05 | FP | NEFP | | | | | | | | |
| | | mM EDTA, 0.02% PS80, | | | | | | | | | | |
| | | pH 6.5 | | | | | | | | | | |
| F16 | 200 | 10 mM PB, 230 mM | SY, SO, | SY, SO, | SY, SO, NEFP | 206.1 | 208.7 | 209.8 | NA | 6.6 | 543 | 39.5 |
| | | Arg · HCl, 0.05 mM EDTA, | FP | FP | | | | | | | | |
| | | 0.02% PS80, 0.1% | | | | | | | | | | |
| | | P188, pH 6.5 | | | | | | | | | | |
| F17 | 200 | 10 mM His, 200 mM | SY, SO, | SY, SO, | SY, SO, FP | 203.0 | 201.3 | 197.1 | NA | 6.6 | 476 | 35.1 |
| | | Arg · HCl, 0.05 mM EDTA, | FP | FP | | | | | | | | |
| | | 0.02% PS80, pH 6.7 | | | | | | | | | | |
| F18 | 200 | 10 mM His, 250 mM | SY, SO, | SY, SO, | SY, SO, FP | 196.1 | 201.4 | 197.8 | NA | 6.6 | 569 | 29.8 |
| | | Arg · HCl, 0.05 mM EDTA, | FP | FP | | | | | | | | |
| | | 0.02% PS80, pH 6.7 | | | | | | | | | | |
| F19 | 210 | 10 mM His, 200 mM | SY, SO, | SY, SO, | SY, SO, FP | 208.7 | 218.2 | 207.6 | NA | 6.6 | 459 | 46.5 |
| | | Arg · HCl, 0.05 mM EDTA, | FP | FP | | | | | | | | |
| | | 0.02% PS80, pH 6.7 | | | | | | | | | | |
| F20 | 210 | 10 mM His, 250 mM | SY, SO, | SY, SO, | SY, SO, FP | 205.4 | 210.3 | 208.6 | NA | 6.6 | 569 | 38.4 |
| | | Arg · HCl, 0.05 mM EDTA, | FP | FP | | | | | | | | |
| | | 0.02% PS80, pH 6.7 | | | | | | | | | | |
| F21 | 200 | 20 mM Histidine, 230 mM | SY, SO, | SY, SO, | SY, SO, FP | 201.8 | 201.9 | 202.6 | 192.4 | 6.8 | 534 | 31.3 |
| | | Arg · HCl, 0.1% P188, pH | FP | FP | | | | | | | | |
| | | 6.8 | | | | | | | | | | |
| 22 | 200 | 20 mM Histidine, 230 mM | SY, SO, | SY, SO, | SY, SO, FP | 202.2 | 202.9 | 200.9 | 192.8 | 7.0 | 547 | 29.2 |
| | | Arg · HCl, 0.1% P188, pH | FP | FP | | | | | | | | |
| | | 7.0 | | | | | | | | | | |

C: Colorless;
CL: Clear liquid;
SO: Slightly opalescent;
SY: Slightly yellow;
Y: Yellow;
FP: Free of visible particles;
EFP: Essentially free of visible particles (no more than 3 visible particles were observed);
NEFP: Not essentially free of visible particles (more than 3 visible particles were observed).
Abbreviations: PB = phosphate buffer

TABLE 10
Break loose and glide force characteristics of formulations

| | | Break loose force and glide force | | |
|---|---|---|---|---|
| | Viscosity | (3 mL Syringe + 25G needle, 140 mm/min) | | |
| | mPa*s- | T0 | | |
| | 20° C. | Break Loose | Average Glide | Max Glide Force(N) |
| No. | T0 | Force(N) | Force (N) | |
| F09 | 49.0 | * | 21.3 | 22.1 |
| F10 | 39.0 | * | 17.7 | 18.6 |
| F11 | 47.7 | * | 21.6 | 22.6 |
| F12 | 33.0 | * | 16.5 | 17.1 |
| F13 | 33.1 | * | 15.7 | 16.3 |
| F14 | 39.9 | * | 19.6 | 20.3 |
| F15 | 36.6 | * | 16.8 | 17.3 |
| F16 | 39.5 | * | 17.7 | 18.3 |
| F17 | 35.1 | * | 16.3 | 16.8 |
| F18 | 29.8 | * | 15.4 | 16.0 |
| F19 | 46.5 | * | 20.4 | 21.2 |
| F20 | 38.4 | * | 17.5 | 18.2 |
| F21 | 31.3 | * | 15.6 | 16.1 |
| F22 | 29.2 | * | 15.8 | 16.4 |

* Due to the high viscosity of solution, the glide force is high and the break loose force could not be determined

For the syringe with 27G needle, the glide force of a solution with a viscosity of 35 cP is less than 20 N.

For the syringe with 25G needle, the glide force of a solution with a viscosity of 39 cP is less than 20 N.

Functional device testing shows that injection force was acceptable for all formulations having a viscosity up to 40-45 cP and an average glide force of 20N. See Table 10.

### Example 3. Further Optimizing High Concentrations of Ab001

### Materials and Methods

Screening for improved viscosity at high Ab001 protein concentration was undertaken. Starting materials was Ab001 at a concentration of 200 mg/mL protein in 20 mM His buffer, 100 mM Arg HCl, pH 6.5. Protein concentration was tested by nanodrop technique, and the method variation was 10%. Osmolality was tested by vapor pressure osmometer.

TABLE 11
Test solutions

| . | Protein | |
|---|---|---|
| | concentration | |
| No | (mg/mL) | Formulation buffer |
| F23 | 200 | 10 mM His, 240 mM Arg, 0.05 mM EDTA, 0.1% |
| | | P188, pH 6.7 |
| F24 | 210 | 10 mM His, 240 mM Arg, 0.05 mM EDTA, 0.1% |
| | | P188, pH 6.7 |
| F25 | 200 | 10 mM His, 240 mM Arg, 0.05 mM EDTA, 0.1% |
| | | P188, pH 7.0 |
| F26 | 210 | 10 mM His, 240 mM Arg, 0.05 mM EDTA, 0.1% |
| | | P188, pH 7.0 |

TABLE 12
Measured Attributes

| attributes | Condition | Sampling Points and Assay | | |
|---|---|---|---|---|
| Thermal | 40° C. | T0 | 3D | 7D |
| | | X, Y | X | X |
| Agitation | 300 rpm, 25° C. | | X | NA |

X=Appearance, protein concentration, SE-UPLC
Y= viscosity, osmolality, LF-NMR

Results. Data were obtained for stability, osmolality, and viscosity for various formulations.

TABLE 13
Stability, Osmolality, and Viscosity Measures

| | | | | | | | | | | | Osmo- | Viscosity |
|---|---|---|---|---|---|---|---|---|---|---|---|---|
| | | | | | | | | | | | lality | mPa*s- |
| | Protein | | Appearance | | | | Protein Conc. | | | | (mOsm/ | 20° C. |
| No. | Conc. | Formulation | T0 | A-3D | 40° C.-3D | 40° C.-7D | T0 | A-3D | 40° C.-3D | 40° C.-7D | kg) | T0 |
| F23 | 200 | 10 mM His, 240 mM | SY, SO, | SY, SO, | SY, SO, FP | SY, SO, FP | 201.4 | 204.4 | 202.9 | 197.8 | 541 | 33.2 |
| | | Arg, 0.05 mM | FP | FP | | | | | | | | |
| | | EDTA, 0.1% | | | | | | | | | | |
| | | P188, pH 6.7 | | | | | | | | | | |
| F24 | 210 | 10 mM His, 240 mM | SY, SO, | SY, SO, | SY, SO, FP | SY, SO, FP | 210.7 | 213.4 | 210.3 | 208.5 | 547 | 41.1 |
| | | Arg, 0.05 mM | FP | FP | | | | | | | | |
| | | EDTA, 0.1% | | | | | | | | | | |
| | | P188, pH 6.7 | | | | | | | | | | |
| F25 | 200 | 10 mM His, 240 mM | SY, SO, | SY, SO, | SY, SO, FP | SY, SO, FP | 205.9 | 205.7 | 209.7 | 200.3 | 554 | 29.1 |
| | | Arg, 0.05 mM | FP | FP | | | | | | | | |
| | | EDTA, 0.1% | | | | | | | | | | |
| | | P188, pH 7.0 | | | | | | | | | | |
| F26 | 210 | 10 mM His, 240 mM | SY, SO, | SY, SO, | SY, SO, FP | SY, SO, FP | 216.3 | 214.5 | 219.2 | 213.0 | 558 | 39.6 |
| | | Arg, 0.05 mM | FP | FP | | | | | | | | |
| | | EDTA, 0.1% | | | | | | | | | | |
| | | P188, pH 7.0 | | | | | | | | | | |

C: Colorless;
CL: Clear liquid;
SO: Slightly opalescent;
SY: Slightly yellow;
Y: Yellow;
FP: Free of visible particles;
EFP: Essentially free of visible particles (no more than 3 visible particles were observed).
A-3D refers to agitation stress at 300 rpm for 3 days at 25 C.

Discussion. This confirmation study established that arginine concentration equal or greater than 200 mM, pH≥6.7, 10 mM to 20 mM His, and 0.1% P188 is a suitable range for viscosity, osmolarity, and appearance (free of particulates). These values were also found to provide chemical stability to the formulations.

These studies revealed the following points:
Histidine selected as buffering agent to maintain pH while preventing HMW/particulate formation. Phosphate buffer resulted in HWS/particulates.
P188 selected as surfactant. Polysorbate PS80 degraded during stability resulting in particulates.
Osmolality of from about 440 to about 540 (e.g., 490) mOsm/kg within reported acceptable range for SC formulations (300-600 mOsm/kg)
pH 6.7 balances high viscosity at low pH and high chemical degradation at high pH
250 mM arginine further reduces viscosity at pH 6-8 (e.g., 6.5 to 7, e.g., 6.8) and stabilizes Ab001.

### Example 4. Stability of Formulations Comprising High Concentrations of Ab001

Stability studies were performed under refrigerated storage (2-8° C.) and different stress conditions (23-27° C., 38-42° C.) on the test formulations (see Table 14). Appearance, pH, protein concentration, SEC-UPLC, CE-SDS-NR & R, iCIEF, HIAC testing were conducted on the samples in this study.

TABLE 14
Test formulations

| | Protein | |
|---|---|---|
| | concentration | |
| No. | (mg/mL) | Formulation |
| A | 180 | 20 mM L-histidine buffer, 200 mM Arginine, 0.05 |
| | | mM EDTA, 0.1% (w/v) P188, at pH 6.7 |
| B | 150 | 20 mM L-histidine buffer, 125 mM Arginine HCI, |
| | | 0.05 mM EDTA, 0.02% (w/v) PS80, at pH 6.0 |

The viscosity Formula B is 13.4 cP at 20° C., and the osmolality is 354 mOsm/kg.

The viscosity Formula A is 19.3 cP at 20° C., and the osmolality is 465 mOsm/kg.

The physical stability of a formulation refers to properties such as color and clarity (appearance), pH, protein aggregation and protein concentration. The presence of visible particulates in solution was determined by visual inspection method. A solution passes visual inspection if it is clear to slightly opalescent, essentially free from visible particulates, and colorless to pale yellow.

The chemical stability refers to the formation of modified forms (e.g., certain types of aggregates, fragments, or charge variant forms) of the antibody. Higher and lower molecular weight degradation products including aggregates and fragments were separated from native antibody by, for example, SE-UPLC and CE-SDS methods. The charge variant forms were separated by iCIEF.

### Formula A

Stability studies were performed under refrigerated storage (2-8° C. for 9 months) and different stress conditions (23-27° C. for 6 months, 38-42° C. for 1 month) on the formulation. Appearance, pH, protein concentration, SEC-UPLC, CE-SDS-NR, CE-SDS-R, iCIEF, HIAC (subvisible particulate matter) testing were conducted on the samples in this study.

The data summary from appearance, protein concentration, pH and subvisible particles (HIAC) for the stability study (5° C. (Upright), 25° C. and 40° C.) as well as the agitation study is shown in Table 15, Table 16, and Table 17.

TABLE 15
Measured attributes at 5° C.

| Test | | T0 | 1M | 2M | 3M | 4M | 6M | 9M |
|---|---|---|---|---|---|---|---|---|
| Color* | | = B5 | = B5 | = B5 | = B5 | = B5 | = B5 | = B5 |
| Clarity (NTU) | | 20.2 | 20.1 | 19.6 | 21.0 | 21.0 | 19.8 | 20.1 |
| Visible Particles | | No visible | No | No | No | No | No | No |
| | | particles | visible | visible | visible | visible | visible | visible |
| | | present | particles | particles | particles | particles | particles | particles |
| | | | present | present | present | present | present | present |
| Sub- | ≥25 μm: | 0 | 0 | 0 | 0 | 0 | 0 | 2 |
| visible | (particles/ | | | | | | | |
| Particulate | container) | | | | | | | |
| Matter | ≥10 μm: | 7 | 15 | 18 | 8 | 5 | 13 | 16 |
| | (particles/ | | | | | | | |
| | container) | | | | | | | |
| | ≥5 μm: | 62 | 80 | 94 | 72 | 56 | 54 | 98 |
| | (particles/ | | | | | | | |
| | container) | | | | | | | |
| | ≥2 μm: | 396 | 432 | 521 | 452 | 401 | 292 | 496 |
| | (particles/ | | | | | | | |
| | container) | | | | | | | |
| Protein Concentration | | 181.4 | 179.0 | 180.0 | 180.7 | 181.8 | 179.7 | 178.7 |
| (mg/mL) | | | | | | | | |
| pH | | 6.7 | 6.6 | 6.7 | 6.7 | 6.7 | 6.7 | 6.7 |
| Charge | Main peak | 70.4 | 70.9 | 70.0 | 71.0 | 70.8 | 70.0 | 70.2 |
| Variants | % | | | | | | | |
| Purity by | Acidic | 18.3 | 18.9 | 20.8 | 20.8 | 20.8 | 21.6 | 22.2 |
| iCIEF | peaks % | | | | | | | |
| | Basic | 11.3 | 10.1 | 9.2 | 8.2 | 8.4 | 8.4 | 7.6 |
| | peaks % | | | | | | | |
| Purity by | Monomer | 99.0 | 99.0 | 98.9 | 98.8 | 98.8 | 98.8 | 98.5 |
| SEC | % | | | | | | | |
| | HMWS % | 1.0 | 0.9 | 1.0 | 1.1 | 1.1 | 1.1 | 1.3 |
| Purity by | Purity % | 98.2 | 97.5 | 98.2 | 97.8 | 97.9 | 97.8 | 97.2 |
| CE-SDS | LMWS% | 1.2 | 1.7 | 1.1 | 1.4 | 1.2 | 1.2 | 2.2 |
| (Reduced) | | | | | | | | |
| Purity by | Purity % | 97.3 | 97.0 | 97.0 | 96.8 | 96.9 | 96.9 | 96.3 |
| CE-SDS | LMWS % | 2.6 | 2.8 | 2.9 | 3.0 | 2.9 | 2.9 | 3.3 |
| (Non- | | | | | | | | |
| reduced) | | | | | | | | |

| | | | | | | | |
|---|---|---|---|---|---|---|---|
| Poloxamer 188 content | 0.089 | 0.103 | 0.104 | 0.104 | 0.107 | 0.104 | 0.097 |
| (w/v %) | | | | | | | |

Abbreviations: NT: Not Tested.
*Color grade as per European Pharmacopeia 2.2.2. in brown color scale

TABLE 16
Measured attributes at 25° C.

| Test | | T0 | 1M | 2M | 3M | 6 M |
|---|---|---|---|---|---|---|
| Color* | | = B5 | = B5 | = B5 | >B5, | >B5, |
| | | | | | <B4 | <B4 |
| Clarity (NTU) | | 20.2 | 20.6 | 19.4 | 20.7 | 21.0 |
| Visible Particles | | No visible | No visible | No visible | No visible | No visible |
| | | particles | particles | particles | particles | particles |
| | | present | present | present | present | present |
| Sub- | ≥25 μm: | 0 | 1 | 1 | 0 | 1 |
| visible | (particles/container) | | | | | |
| Particulate | ≥10 μm: | 7 | 8 | 35 | 30 | 32 |
| Matter | (particles/container) | | | | | |
| | ≥5 μm: | 62 | 61 | 205 | 208 | 192 |
| | (particles/container) | | | | | |
| | ≥2 μm: | 396 | 430 | 924 | 922 | 884 |
| | (particles/container) | | | | | |
| Protein Concentration (mg/mL) | | 181.4 | 178.9 | 180.8 | 182.5 | 178.4 |
| pH | | 6.7 | 6.7 | 6.7 | 6.7 | 6.7 |
| Charge | Main peak % | 70.4 | 67.8 | 62.8 | 59.0 | 48.1 |
| Variants | Acidic peaks % | 18.3 | 24.0 | 29.5 | 34.4 | 45.2 |
| Purity by | Basic peaks % | 11.3 | 8.2 | 7.6 | 6.6 | 6.7 |
| iCIEF | | | | | | |
| Purity by | Monomer % | 99.0 | 98.8 | 98.5 | 98.1 | 97.2 |
| SEC | HMWS % | 1.0 | 1.1 | 1.2 | 1.5 | 1.9 |
| Purity by | Purity % | 98.2 | 97.0 | 97.3 | 96.6 | 95.6 |
| CE-SDS | LMWS % | 1.2 | 2.1 | 1.7 | 2.4 | 3.0 |
| (Reduced) | | | | | | |
| Purity by | Purity % | 97.3 | 96.6 | 96.1 | 95.3 | 93.9 |
| CE-SDS | LMWS % | 2.6 | 3.2 | 3.6 | 4.3 | 5.7 |
| (Non- | | | | | | |
| reduced) | | | | | | |

| | | | | | |
|---|---|---|---|---|---|
| Poloxamer 188 content (w/v %) | 0.089 | 0.100 | 0.102 | 0.103 | 0.102 |

Abbreviations: NT: Not Tested.
*Color grade as per European Pharmacopeia 2.2.2. in brown color scale

TABLE 17
Measured attributes at 40° C.

| Test | | T0 | 1 Week | 2 Weeks | 1 Months |
|---|---|---|---|---|---|
| Color* | | =B5 | =B5 | >B5, <B4 | >B5, <B4 |
| Clarity (NTU) | | 20.2 | 20.1 | 19.9 | 20.1 |
| Visible Particles | | No | No | No | No |
| | | visible | visible | visible | visible |
| | | particles | particles | particles | particles |
| | | present | present | present | present |
| Sub- | ≥25 μm: | 0 | 0 | 0 | 0 |
| | (particles/ | | | | |
| | container) | | | | |
| visible | ≥10 μm: | 7 | 8 | 6 | 13 |
| | (particles/ | | | | |
| | container) | | | | |
| Particulate | ≥5 μm: | 62 | 58 | 55 | 89 |
| | (particles/ | | | | |
| | container) | | | | |
| Matter | ≥2 μm: | 396 | 313 | 305 | 46 |
| | (particles/ | | | | |
| | container) | | | | |
| Protein Concentration (mg/ | | 181.4 | 181.3 | 180.1 | 178.7 |
| mL) | | | | | |
| pH | | 6.7 | 6.7 | 6.7 | 6.7 |
| Charge | Main peak % | 70.4 | 63.6 | 59.2 | 46.8 |
| Variants | | | | | |
| Purity by | Acidic peaks | 18.3 | 26.4 | 32.0 | 45.6 |
| iCIEF | % | | | | |
| | Basic peaks % | 11.3 | 10.1 | 8.8 | 7.7 |
| Purity by | Monomer % | 99.0 | 98.3 | 98.0 | 96.9 |
| SEC | HMWS % | 1.0 | 1.4 | 1.4 | 2.0 |
| Purity by | Purity % | 98.2 | 97.3 | 96.9 | 94.7 |
| CE-SDS | | | | | |
| (Reduced) | LMWS % | 1.2 | 1.9 | 2.3 | 3.9 |
| Purity by | Purity % | 97.3 | 96.4 | 95.4 | 92.6 |
| CE-SDS | LMWS % | 2.6 | 3.4 | 4.3 | 6.8 |
| (Non- | | | | | |
| reduced) | | | | | |

| | | | | |
|---|---|---|---|---|
| Poloxamer 188 content | 0.089 | NT | NT | NT |
| (w/v %) | | | | |

Abbreviations: NT: Not Tested.
*Color grade as per European Pharmacopeia 2.2.2. in brown color scale

### Results:

All samples remained slightly brown, slightly opalescent, and essentially free of visible particles in the formulation under all testing conditions in this study. The purity measured by CE-SDS-NR & CE-SDS-R, and the measured main peak % from the SEC-UPLC and iCIEF analyses declined slightly following incubation under stress condition of 40° C. for 1 month. Furthermore, the testing results also indicated no obvious effect on the stability under agitation condition.

Based on these results, the formulation is heat stable at 25° C. for at least six months. The formulation is stable at the long-term conditions of 2-8° C.

### Formula B

Stability studies were performed under refrigerated storage (2-8° C. for 18 months) and different stress conditions (23-27° C. for 6 months, 38-42° C. for 1 month) on the formulation. Appearance, pH, protein concentration, SEC-UPLC, CE-SDS-NR & CE-SDS-R, iCIEF, HIAC (subvisible particulate matter), testing were conducted on the samples in this study.

TABLES 18, 19 and 20
show measured attributes at 5° C. (Upright), 25° C. and 40° C..
Table 18. Measured attributes at 5° C.

| Test | | T0 | 1M | 2M | 3M | 4M | 6M | 9M | 12M | 18M |
|---|---|---|---|---|---|---|---|---|---|---|
| Color | | = B5 | = B5 | = B5 | =B5 | =B5 | = B5 | >B5, <B4 | >B5, <B4 | >B5, <B4 |
| Clarity (NTU) | | 28.1 | 27.9 | 28.2 | 26.9 | 29.0 | 30.5 | 27.0 | 28.9 | 26.1 |
| Visible Particles | | No visible | No visible | No visible | No visible | No visible | No visible | No visible | No visible | No visible |
| | | particles | particles | particles | particles | particles | particles | particles | particles | particles |
| | | present | present | present | present | present | present | present | present | present |
| Sub- | ≥25 μm: | 0 | 0 | 0 | 4 | 2 | 2 | 1 | 0 | 2 |
| visible | (particles/container) | | | | | | | | | |
| Particulate | ≥10 μm: | 12 | 6 | 10 | 12 | 6 | 23 | 9 | 26 | 10 |
| Matter | (particles/container) | | | | | | | | | |
| | ≥5 μm: | 116 | 70 | 38 | 61 | 80 | 141 | 116 | 130 | 96 |
| | (particles/container) | | | | | | | | | |
| | ≥2 μm: | 763 | 536 | 380 | 462 | 679 | 909 | 854 | 557 | 926 |
| | (particles/container) | | | | | | | | | |
| Protein Concentration (mg/mL) | | 154.6 | 153.2 | 151.3 | 152.1 | 152.5 | 153.3 | 152.1 | 151.5 | 151.1 |
| pH | | 6.0 | 6.1 | 6.0 | 6.0 | 6.0 | 6.0 | 6.0 | 6.0 | 6.0 |
| Charge | Main peak % | 68.6 | 69.7 | 70. | 69.7 | 70.3 | 68.6 | 68.0 | 69.1 | 68.3 |
| Variants | Acidic peaks % | 19.2 | 19.0 | 17.5 | 19.1 | 18.2 | 19.1 | 20.3 | 19.9 | 20.4 |
| Purity by | Basic peaks % | 12.2 | 11.3 | 12.2 | 11.2 | 11.6 | 12.3 | 11.7 | 11.1 | 11.3 |
| iCIEF | | | | | | | | | | |
| Purity by | Monomer % | 99.1 | 99.1 | 99.0 | 98.9 | 98.8 | 98.3 | 98.7 | 98.6 | 98.4 |
| SEC | HMWS % | 0.8 | 0.9 | 0.9 | 1.0 | 1.1 | 1.5 | 1.2 | 1.3 | 1.4 |
| Purity by | Purity % | 98.3 | 98.5 | 97.9 | 97.2 | 98.2 | 97.9 | 97.8 | 98.1 | 97.4 |
| CE-SDS | LMWS% | 1.1 | 0.9 | 1.3 | 1.6 | 1.4 | 1.5 | 1.4 | 1.2 | 1.9 |
| (Reduced) | | | | | | | | | | |
| Purity by | Purity % | 97.0 | 97.0 | 96.8 | 96.6 | 96.8 | 96.5 | 96.8 | 96.7 | 96.4 |
| CE-SDS | LMWS % | 3.0 | 2.9 | 2.9 | 3.0 | 3.0 | 3.2 | 3.0 | 3.1 | 3.1 |
| (Non- | | | | | | | | | | |
| reduced) | | | | | | | | | | |

| | | | | | | | | | |
|---|---|---|---|---|---|---|---|---|---|
| Polysorbate 80 (% w/v) | 0.018 | 0.018 | 0.018 | 0.017 | 0.017 | 0.016 | 0.016 | 0.018 | 0.017 |

TABLE 19
Measured attributes at 25° C.

| Test | | T0 | 1 M | 2 M | 3 M | 6 M |
|---|---|---|---|---|---|---|
| Color | | =B5 | =B5 | >B5 | >B5 | <B4 |
| Clarity (NTU) | | 28.1 | 27.7 | 27.9 | 27.5 | 30.2 |
| Visible Particles | | No | No visible | No visible | No visible | No visible |
| | | visible | particles | particles | particles | particles |
| | | particles | present | present | present | present |
| | | present | | | | |
| Sub- | ≥25 μm: | 0 | 0 | 0 | 3 | 9 |
| | (particles/container) | | | | | |
| visible | ≥10 μm: | 12 | 8 | 2 | 8 | 30 |
| | (particles/container) | | | | | |
| Particulate | ≥5 μm: | 116 | 101 | 81 | 110 | 144 |
| | (particles/container) | | | | | |
| Matter | ≥2 μm: | 763 | 906 | 777 | 856 | 863 |
| | (particles/container) | | | | | |
| Protein Concentration (mg/mL) | | 154.6 | 151.0 | 151.9 | 151.6 | 150.1 |
| pH | | 6.0 | 6.1 | 6.1 | 6.0 | 6.0 |
| Charge | Main peak % | 68.6 | 64.0 | 60.5 | 58.5 | 52.8 |
| Variants | | | | | | |
| Purity by | Acidic peaks % | 19.2 | 20.0 | 20.7 | 23.9 | 30.3 |
| iCIEF | Basic peaks % | 12.2 | 16.0 | 18.9 | 17.6 | 16.9 |
| Purity by | Monomer % | 99.1 | 98.8 | 98.6 | 98.2 | 96.6 |
| SEC | HMWS % | 0.8 | 1.1 | 1.2 | 1.5 | 2.7 |
| Purity by | Purity % | 98.3 | 97.8 | 97.3 | 96.6 | 96.2 |
| CE-SDS | | | | | | |
| (Reduced) | LMWS% | 1.1 | 1.5 | 1.7 | 1.9 | 2.9 |
| Purity by | Purity % | 97.0 | 96.6 | 96.0 | 95.6 | 93.5 |
| CE-SDS | | | | | | |
| (Non- | LMWS% | 3.0 | 3.2 | 3.6 | 3.9 | 5.5 |
| reduced) | | | | | | |

| | | | | | |
|---|---|---|---|---|---|
| Polysorbate 80 (% w/v) | 0.018 | 0.018 | 0.018 | 0.017 | 0.015 |

TABLE 20
Measured attribues at 40° C.

| Test | | T0 | 1 W | 2 W | 4 W |
|---|---|---|---|---|---|
| Color | | =B5 | =B5 | =B5 | >B5 |
| Clarity (NTU) | | 28.1 | 27.2 | 27.8 | 27.9 |
| Visible Particles | | No | No | No | No |
| | | visible | visible | visible | visible |
| | | particles | particles | particles | particles |
| | | present | present | present | present |
| | ≥25 μm: | 0 | 1 | 0 | 0 |
| | (particles/ | | | | |
| | container) | | | | |
| Sub-visible | ≥10 μm: | 12 | 10 | 8 | 12 |
| | (particles/ | | | | |
| | container) | | | | |
| Particulate | ≥5 μm: | 116 | 70 | 73 | 125 |
| | (particles/ | | | | |
| | container) | | | | |
| Matter | ≥2 μm: | 763 | 536 | 686 | 954 |
| | (particles/ | | | | |
| | container) | | | | |
| Protein Concentration (mg/ | | 154.6 | 151.5 | 154.3 | 153.4 |
| mL) | | | | | |
| pH | | 6.0 | 6.1 | 6.1 | 6.0 |
| Charge | Main peak % | 68.6 | 56.9 | 50.1 | 43.8 |
| Variants | Acidic peaks % | 19.2 | 20.4 | 23.7 | 29.1 |
| Purity by | Basic peaks % | 12.2 | 22.7 | 26.2 | 27.1 |
| iCIEF | | | | | |
| Purity by | Monomer % | 99.1 | 98.6 | 98.2 | 97.3 |
| SEC | HMWS % | 0.8 | 1.2 | 1.5 | 2.1 |
| Purity by | Purity % | 98.3 | 97.6 | 97.4 | 96.1 |
| CE-SDS | LMWS% | 1.1 | 2.0 | 1.9 | 3.0 |
| (Reduced) | | | | | |
| Purity by | Purity % | 97.0 | 96.6 | 94.7 | 93.9 |
| CE-SDS | LMWS% | 3.0 | 3.2 | 4.7 | 5.6 |
| (Non- | | | | | |
| reduced) | | | | | |

### Results:

All samples remained slightly yellow, slightly opalescent, and essentially free of visible particles in the formulation under all testing conditions in this study. The purity measured by CE-SDS-NR & CE-SDS-R, and the measured main peak % from the SEC-UPLC and iCIEF analyses declined slightly following incubation at 40° C. for 1 month and incubation at 25° C. for 6 months.

Furthermore, the testing results also indicated no obvious effect on the stability under agitation condition.

Based on these results, the formulation is heat stable at 25° C. for at least six months. The formulation is stable at the long-term conditions of 2-8° C.

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

We claim:

1. A pharmaceutical aqueous formulation comprising:
a. an anti-α4β7 antibody at a concentration in a range of from about 160 mg/mL to about 300 mg/mL;
b. a buffer selected from phosphate buffer, Tris, and histidine at a concentration of from about 10 mM to about 20 mM; and
c. a chelating agent; and
d. a surfactant,
wherein the pharmaceutical aqueous formulation has a pH ranging from about 6 to about 8,
an osmolality ranging from about 400 mOsm/kg to about 600 mOsm/kg, and a viscosity ranging from about 10 cP to about 25 cP at 20° C.,
wherein the anti-α4β7 antibody has a solubility ranging from about 100 mg/mL to about 300 mg/mL in the buffer at 30° C.,
wherein the anti-α4β7 antibody comprises a modified Fc and has an extended half-life compared to the half-life of a comparable anti-α4β7 antibody comprising a wild-type Fc region, and
wherein the pharmaceutical aqueous formulation is heat stable at about 25° C. for at least 6 months.

2. The pharmaceutical aqueous formulation of claim 1, wherein the anti-α4β7 antibody concentration is at least about 180 mg/mL.

3. The pharmaceutical aqueous formulation of claim 1, wherein the anti-α4β7 antibody comprises a heavy chain polypeptide consisting of an amino acid sequence according to SEQ ID NO: 1 and a light chain polypeptide consisting of an amino acid sequence according to SEQ ID NO: 3.

4. The pharmaceutical aqueous formulation of claim 1, wherein the pH ranges from about 6.5 to about 7.5.

5. The pharmaceutical aqueous formulation of claim 1 further comprising arginine or arginine-HCl.

6. The pharmaceutical aqueous formulation of claim 5, wherein the arginine or arginine-HCl at a concentration of from about 200 mM to about 250 mM.

7. The pharmaceutical aqueous formulation of claim 1, wherein the chelating agent is EDTA.

8. The pharmaceutical aqueous formulation of claim 7, wherein the EDTA is present at a concentration of about 0.05 mM.

9. The pharmaceutical aqueous formulation of claim 1, wherein the surfactant is polysorbate-80 or poloxamer 188.

10. The pharmaceutical aqueous formulation of claim 9, wherein the surfactant is poloxamer 188 present at a concentration of (w/v) 0.1%.

11. The pharmaceutical aqueous formulation of claim 1, wherein the osmolality is about 500 mOsm/kg.

12. The pharmaceutical aqueous formulation of claim 1, wherein the buffer is L-histidine.

13. The pharmaceutical aqueous formulation of claim 12, wherein the L-histidine is present at a concentration of about 20 mM.

14. The pharmaceutical aqueous formulation of claim 1, wherein the pharmaceutical aqueous formulation is for subcutaneous administration.

15. The pharmaceutical aqueous formulation of claim 1, wherein the pharmaceutical aqueous formulation is stable at about 5° C. for at least 9 months.

16. The pharmaceutical aqueous formulation of claim 1, wherein the pharmaceutical aqueous formulation is an injectable pharmaceutical aqueous formulation.

17. A method of treating a gastrointestinal inflammatory disease comprising administering to a subject in need thereof the pharmaceutical aqueous formulation of claim 1.

18. The method of claim 17, wherein the gastrointestinal inflammatory disease is inflammatory bowel disease.

19. The method of claim 18, wherein the inflammatory bowel disease is ulcerative colitis.

20. The method of claim 18, wherein the inflammatory bowel disease is Crohn's disease.

21. The method of claim 17, wherein the pharmaceutical aqueous formulation is administered at a lower volume compared to a pharmaceutical aqueous formulation comprising a comparable anti-α4β7 antibody with a wild-type Fc region.

22. The method of claim 21, wherein the pharmaceutical aqueous formulation is administered subcutaneously.

23. The pharmaceutical aqueous formulation of claim 3, wherein the anti-α4β7 antibody has a glycosylation pattern comprising a proportion of GOF from about 55.1% to about 60.4%.

24. The pharmaceutical aqueous formulation of claim 1, wherein the pharmaceutical aqueous formulation exhibits a glide force of less than 20 N when injected through 25G needle.

25. The pharmaceutical aqueous formulation of claim 1, wherein a glide force of the pharmaceutical aqueous formulation for subcutaneous injection is less than 20 N.

26. A prefilled syringe comprising a pharmaceutical aqueous formulation, wherein the pharmaceutical aqueous formulation comprises:
a. an anti-α4β7 antibody at a concentration in a range of from about 160 mg/mL to about 300 mg/mL;
b. a buffer selected from phosphate buffer, Tris, and histidine at a concentration of from about 10 mM to about 20 mM; and
c. a chelating agent; and
d. a surfactant,
wherein the pharmaceutical aqueous formulation has a pH ranging from about 6 to about 8, an osmolality ranging from about 400 mOsm/kg to about 600 mOsm/kg, and a viscosity ranging from about 10 cP to about 25 cP at 20° C.,
wherein the anti-α4β7 antibody has a solubility ranging from about 100 mg/mL to about 300 mg/mL at 30° C.,
wherein the anti-α4β7 antibody comprises a modified Fc and has an extended half-life compared to the half-life of a comparable anti-α4β7 antibody comprising a wild-type Fc region, and
wherein the pharmaceutical aqueous formulation is heat stable at about 25° C. for at least 6 months, and
wherein a glide force of the pharmaceutical aqueous formulation is less than 20 N when injected through 25G needle.

27. An auto-injector comprising a pharmaceutical aqueous formulation for subcutaneous injection, wherein the pharmaceutical aqueous formulation comprises
a. an anti-α4β7 antibody at a concentration in a range of from about 160 mg/mL to about 300 mg/mL;
b. a buffer selected from phosphate buffer, Tris, and histidine at a concentration of from about 10 mM to about 20 mM; and
c. a chelating agent; and
d. a surfactant,
wherein the pharmaceutical aqueous formulation has a pH ranging from about 6 to about 8, an osmolality ranging from about 400 mOsm/kg to about 600 mOsm/kg, and a viscosity ranging from about 10 cP to about 25 cP at 20° C.,
wherein the anti-α4β7 antibody has a solubility ranging from about 100 mg/mL to about 300 mg/mL at 30° C.,
wherein the anti-α4β7 antibody comprises a modified Fc and has an extended half-life compared to the half-life of a comparable anti-α4β7 antibody comprising a wild-type Fc region,
wherein the pharmaceutical aqueous formulation is heat stable at about 25° C. for at least 6 months, and
wherein a glide force of the pharmaceutical aqueous formulation is less than 20 N.
